(19) United States
(12) Patent Application Publication
(10) Pub. No.: US 2026/0234183 A1
(43) Pub. Date: Aug. 13, 2026

(54) SOLID STATE FORMS OF FOSGONIMETON AND SALTS THEREOF

(71) Applicant: ASSIA CHEMICAL INDUSTRIES LTD., Tel Aviv (IL)

(72) Inventors: Ivana Landeka, Zagreb (HR); Klara Karadakic, Sesvete (HR); Dijana Skalec Samec, Jastrebarsko (HR)

(21) Appl. No.: 19/475,188

(22) PCT Filed: Apr. 17, 2024
(86) PCT No.: PCT/IB2024/053749

§ 371 Date: Oct. 14, 2025

Related U.S. Application Data
(60) Provisional application No. 63/460,073, filed on Apr. 18, 2023.
(60) Provisional application No. 63/472,857, filed on Jun. 14, 2023.

(51) Int. Cl. C07K 5/06; A61K 38/00

(52) U.S. Cl. CPC C07K 5/06191; A61K 38/00

(57) ABSTRACT

The present disclosure encompasses solid state forms of Fosgonimeton and salts thereof, and pharmaceutical compositions thereof.

### FIELD OF THE DISCLOSURE

The present disclosure encompasses solid state forms of Fosgonimeton and salts thereof, and pharmaceutical compositions thereof.

### BACKGROUND OF THE DISCLOSURE

Fosgonimeton, [4-[(2S)-3-[[(2S,3S)-1-[(6-amino-6-oxohexyl)amino]-3-methyl-1-oxopentan-2-yl]amino]-2-(hexanoylamino)-3-oxopropyl]phenyl]dihydrogen phosphate, has the following chemical structure:

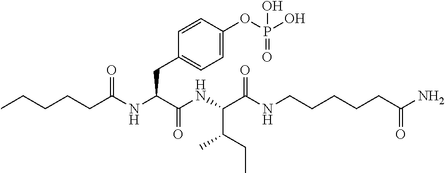 [SMILES: CCCCCC(=O)N[C@@H](Cc1ccc(OP(=O)(O)O)cc1)C(=O)N[C@H](C(=O)NCCCCCC(N)=O)[C@@H](C)CC]

Fosgonimeton is an investigational compound in clinical development for the treatment of patients with Alzheimer's Disease, Parkinson's disease (PD), Lewy body disease or disorder(s) (LBD), Parkinson's Disease Dementia (PDD) and/or dementia with Lewy bodies (DLB), cognitive impairment, depression, schizophrenia, Diabetic Peripheral Neuropathic pain and/or Post-Herpetic Neuralgia.

The compound, a diammonium salt, and a sodium salt are described in International Publication No. WO 2017/210489.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Fosgonimeton.

### SUMMARY OF THE DISCLOSURE

The present disclosure provides crystalline polymorphs of Fosgonimeton, potassium salts and sodium salts thereof, co-crystals of Fosgonimeton, and pharmaceutical compositions thereof. Any one of the crystalline polymorphs can be used to prepare other solid state forms of Fosgonimeton, Fosgonimeton salts and/or co-crystals thereof and their solid state forms.

The present disclosure also provides uses of said solid state forms of Fosgonimeton or salts and/or cocrystals thereof in the preparation of other solid state forms of Fosgonimeton or salts thereof.

The present disclosure provides crystalline forms of Fosgonimeton or salts and/or cocrystals thereof for use in medicine, particularly for the treatment of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and/or schizophrenia; and more particularly for the treatment of patients with Alzheimer's Disease, Parkinson's disease (PD), Lewy body disease or disorder(s) (LBD), Parkinson's Disease Dementia (PDD) and/or dementia with Lewy bodies (DLB).

The present disclosure also encompasses the use of any one or a combination of the crystalline polymorphs of Fosgonimeton or the salts thereof and/or cocrystals thereof of the present disclosure for the preparation of pharmaceutical compositions and/or formulations.

In another aspect, the present disclosure provides pharmaceutical compositions comprising any one of or a combination of the crystalline polymorphs of Fosgonimeton or salts and/or cocrystals thereof according to the present disclosure.

The present disclosure includes processes for preparing the above mentioned pharmaceutical compositions. The processes include combining any one or a combination of the crystalline polymorphs of Fosgonimeton or salts and/or cocrystals thereof with at least one pharmaceutically acceptable excipient.

The crystalline polymorphs of Fosgonimeton as defined herein and the pharmaceutical compositions or formulations of the crystalline polymorph of Fosgonimeton or salts and/or cocrystals thereof may be used as medicaments, particularly for the treatment of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and/or schizophrenia; and more particularly for the treatment of Alzheimer's Disease, Parkinson's disease (PD), Lewy body disease or disorder(s) (LBD), Parkinson's Disease Dementia (PDD) and/or dementia with Lewy bodies (DLB).

The present disclosure also provides methods for the treatment of patients with Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and/or schizophrenia; and more particularly Alzheimer's Disease, Parkinson's disease (PD), Lewy body disease or disorder(s) (LBD), Parkinson's Disease Dementia (PDD) and/or dementia with Lewy bodies (DLB), by administering a therapeutically effective amount of any one or a combination of the crystalline polymorphs of Fosgonimeton or salts thereof of the present disclosure, or at least one of the above pharmaceutical compositions, to a subject suffering from Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and/or schizophrenia; and more particularly Alzheimer's Disease, Parkinson's disease (PD), Lewy body disease or disorder(s) (LBD), Parkinson's Disease Dementia (PDD) and/or dementia with Lewy bodies (DLB) or otherwise in need of the treatment.

The present disclosure also provides uses of crystalline polymorphs of Fosgonimeton or salts and/or cocrystals thereof of the present disclosure, or at least one of the above pharmaceutical compositions, for the manufacture of medicaments for treating e.g., Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and/or schizophrenia; and more particularly patients with Alzheimer's Disease, Parkinson's disease (PD), Lewy body disease or disorder(s) (LBD), Parkinson's Disease Dementia (PDD) and/or dementia with Lewy bodies (DLB).

### BRIEF DESCRIPTION OF THE DRAWINGS

FIG. 1 shows a characteristic X-ray powder diffraction pattern (XRPD) of Fosgonimeton Form 1.

FIG. 2A shows the X-ray powder diffraction pattern (XRPD) of Fosgonimeton mono potassium salt form K1 prepared according to Example 2, procedure A.

FIG. 2B shows the X-ray powder diffraction pattern (XRPD) of Fosgonimeton mono potassium salt form K1 prepared according to Example 2, procedure B.

FIG. 3 shows the X-ray powder diffraction pattern (XRPD) of Fosgonimeton mono sodium salt form N2.

FIG. 4 shows the X-ray powder diffraction pattern (XRPD) of Fosgonimeton Cysteine form C1.

FIG. 5 shows the X-ray powder diffraction pattern (XRPD) of Fosgonimeton mono sodium salt form N5.

FIG. 6 shows the overlay of the X-ray powder diffraction pattern (XRPD) of Fosgonimeton mono sodium salt obtained according to example 6 and the X-ray powder diffraction pattern (XRPD) of Fosgonimeton mono sodium salt form N2.

FIG. 7 shows the solid state ¹³C-NMR of Fosgonimeton form 1 (200-0 ppm).

FIG. 7A shows the solid state ¹³C-NM/IR of Fosgonimeton form 1 (200-100 ppm).

FIG. 7B shows the solid state ¹³C-NMR of Fosgonimeton form 1 (100-0 ppm).

FIG. 8 shows the solid state ¹³C-NM/IR of Fosgonimeton monosodium salt form N2 (200-0 ppm).

FIG. 8A shows the solid state ¹³C-NMR of Fosgonimeton monosodium salt form N2 (200-100 ppm).

FIG. 8B shows the solid state ¹³C-NMR of Fosgonimeton monosodium salt form N2 (100-0 ppm).

FIG. 9 shows the X-ray powder diffraction pattern (XRPD) of amorphous Fosgonimeton.

### DETAILED DESCRIPTION OF THE DISCLOSURE

The present disclosure encompasses a crystalline form of Fosgonimeton and a crystalline form of Fosgonimeton, potassium and sodium salts thereof, co-crystals of Fosgonimeton and pharmaceutical compositions thereof.

Solid state properties of Fosgonimeton and crystalline polymorphs thereof can be influenced by controlling the conditions under which Fosgonimeton and crystalline polymorphs thereof are obtained in solid form.

The solid state forms of Fosgonimeton (e.g. Fosgonimeton, a Fosgonimeton salt) as described in any aspect or embodiment of the present disclosure may be polymorphically pure, or substantially free of any other solid state (or polymorphic) forms.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. For example, polymorphically pure Fosgonimeton form 1 means that the solid state form is substantially free of other solid state forms of Fosgonimeton. Thus, a crystalline polymorph of Fosgonimeton or a salt described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Fosgonimeton, salt or cocrystal. In some embodiments of the disclosure, the described crystalline polymorph of Fosgonimeton, salt or cocrystal may contain from about 1% to about 20% (w/w), from about 5% to about 20% (w/w), or from about 5% to about 10% (w/w) of one or more other crystalline polymorph of the same Fosgonimeton.

Depending on which other crystalline polymorphs a comparison is made, the crystalline polymorphs of Fosgonimeton, salts or cocrystals of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. Particularly, the crystalline forms of Fosgonimeton, Fosgonimeton sodium salt, Fosgonimeton potassium salt, as described in any aspect or embodiment of the present disclosure, may be stable, for example to conditions of high relative humidity, may be thermally stable and/or may have advantageous hygroscopicity properties (e.g. non-hygroscopic or low hygroscopicity). Crystalline forms N2 and N5 of Fosgonimeton sodium salt, crystalline form K1 of Fosgonimeton potassium salt and crystalline form 1 of Fosgonimeton may be especially stable to conditions of high humidity. Crystalline forms N2 and N5 of Fosgonimeton sodium salt, crystalline form K1 of Fosgonimeton potassium salt and crystalline form 1 of Fosgonimeton may be non-hygroscopic or have low hygroscopicity. Crystalline form N2 of Fosgonimeton sodium salt, crystalline form K1 of Fosgonimeton potassium salt and crystalline form 1 of Fosgonimeton may be especially thermally stable. Crystalline forms N2 and N5 of Fosgonimeton sodium salt, crystalline form K1 of Fosgonimeton potassium salt and crystalline form 1 of Fosgonimeton may exhibit good stability under stress conditions.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Fosgonimeton (e.g. Fosgonimeton, Fosgonimeton salts) referred to herein as being characterized by graphical data “as depicted in” or “as substantially depicted in” a Figure will thus be understood to include any crystal forms of Fosgonimeton characterized with the graphical data having such small variations, as are well known to the skilled person, in comparison with the Figure.

As used herein, and unless stated otherwise, the term “anhydrous” in relation to crystalline forms of Fosgonimeton relates to a crystalline form of Fosgonimeton which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, unless stated otherwise, unit cell information was obtained by solving the crystal structure.

“Co-Crystal” or “Co-crystal” as used herein is defined as a crystalline material including two or more molecules in the same crystalline lattice and associated by non-ionic and non-covalent bonds. In some embodiments, the co-crystal includes two molecules which are in natural state.

As used herein, the term “isolated” in reference to crystalline polymorph of Fosgonimeton of the present disclosure corresponds to a crystalline polymorph of Fosgonimeton that is physically separated from the reaction mixture in which it is formed.

As used herein, unless stated otherwise, the XRPD measurements are taken using copper K α radiation wavelength 1.54184 Å. XRPD peaks reported herein are optionally measured using CuK α radiation, λ=1.54184 Å, typically at a temperature of 25±3° C.

As used herein, unless stated otherwise, the ¹³C CP/MAS spectra employing cross-polarization were acquired using the standard cross-polarization pulse scheme at spinning frequency 15 kHz, optionally using a 500 MHz NMR instrument.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The solid state forms of Fosgonimeton (e.g. Fosgonimeton, Fosgonimeton salts) as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w) or less, about 0.8% (w/w) or less, about 0.6% (w/w) or less, about 0.4% (w/w) or less, about 0.2% (w/w) or less, about 0.1% (w/w) or less, or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Fosgonimeton, salts or co-crystal thereof or Fosgonimeton intermediate described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2% (w/w), greater than about 99.4% (w/w), greater than about 99.6% (w/w), greater than about 99.8% (w/w), greater than about 99.9% (w/w), or about 100% of the subject Fosgonimeton, salts or co-crystal thereof or Fosgonimeton intermediate. Alternatively, pure or purified Fosgonimeton, salts or co-crystals thereof, or a Fosgonimeton intermediate, described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2% area percent, greater than about 99.4% area percent, greater than about 99.6% area percent, greater than about 99.8% area percent, greater than about 99.9% area percent, or about 100% of the subject Fosgonimeton salts or co-crystal thereof or Fosgonimeton intermediate.

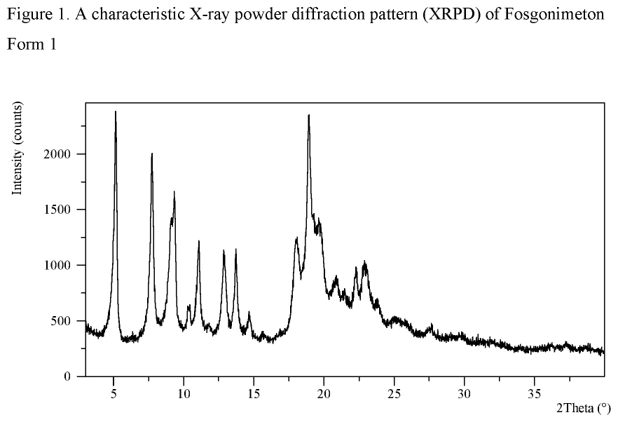

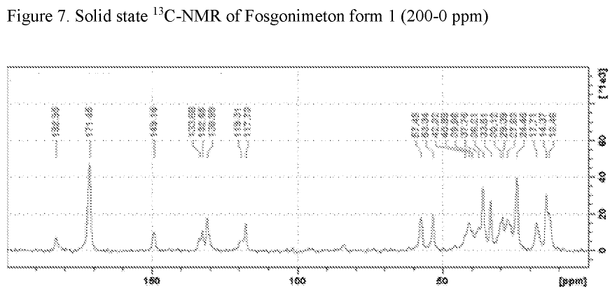

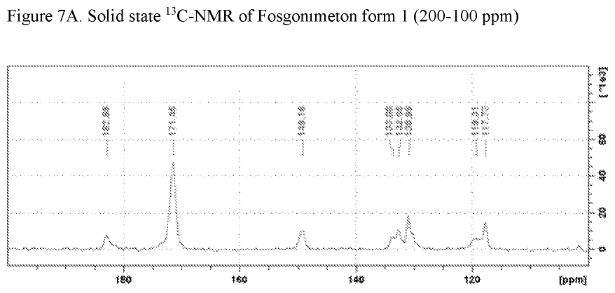

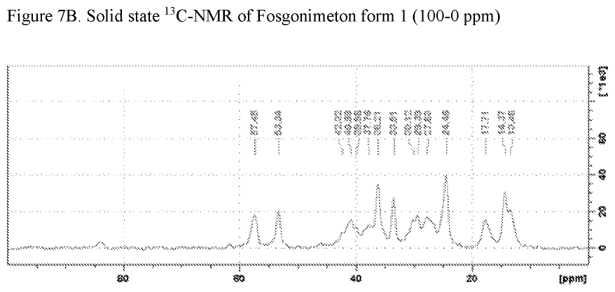

The present disclosure includes a crystalline polymorph Fosgonimeton designated Form 1. The crystalline Form 1 of Fosgonimeton may be characterized by data selected from one or more of the following: an X-ray powder diffraction pattern substantially as depicted in FIG. 1; an X-ray powder diffraction pattern having peaks at 5.2, 9.4 and 12.9 degrees 2-theta±0.2 degrees 2-theta; a solid state ¹³C NMR spectrum having characteristic peaks at 171.5, 149.2, 117.7, 57.5 and 33.5 ppm±0.2 ppm; a solid state ¹³C NMR spectrum having the following chemical shift absolute differences from reference peak at 24.5 ppm±0.2 ppm: 147.0, 124.7, 93.2, 33.0 and 9.0 ppm±0.1 ppm; a solid state ¹³C NMR spectrum substantially as depicted in FIG. 7, 7A or 7B; and combinations of these data.

Crystalline Form 1 of Fosgonimeton may be further characterized by an X-ray powder diffraction pattern having peaks at 5.2, 9.4 and 12.9 degrees 2-theta±0.2 degrees 2-theta, and also having any one or both additional peaks selected from 13.7 and 18.9 degrees 2-theta±0.2 degrees 2-theta.

Crystalline Form 1 of Fosgonimeton may be alternatively characterized by an X-ray powder diffraction pattern having peaks at 5.2, 9.4, 12.9, 13.7 and 18.9 degrees 2-theta±0.2 degrees 2-theta.

Crystalline Form 1 of Fosgonimeton may be further characterized by an X-ray powder diffraction pattern having peaks at 5.2, 9.4, 12.9, 13.7 and 18.9 degrees 2-theta±0.2 degrees 2-theta, and also having any one, two, three, four or five additional peaks selected from 7.7, 11.1, 17.9, 19.8 and 22.3 degrees 2-theta±0.2 degrees 2-theta.

Crystalline Form 1 of Fosgonimeton may be alternatively characterized by an X-ray powder diffraction pattern having peaks at 5.2, 7.7, 9.4, 11.1, 12.9, 13.7, 17.9, 18.9, 19.8 and 22.3 degrees 2-theta±0.2 degrees 2-theta.

According to any aspect or embodiment of the disclosure crystalline Fosgonimeton Form 1 may be additionally characterized by an X-ray powder diffraction pattern having an absence of peaks at one or more of the following: 3.0 to 4.2 degrees 2-theta±0.2 degrees 2-theta or 3.5 to 4.0 degrees 2-theta±0.2 degrees 2-theta; 6.0 to 7.2 degrees 2-theta±0.2 degrees 2-theta; 8.3 to 8.6 degrees 2-theta±0.2 degrees 2-theta; and 16.4 to 17.0 degrees 2-theta±0.2 degrees 2-theta. Particularly, crystalline Fosgonimeton Form 1 may be characterized by an X-ray powder diffraction pattern according to any aspect or embodiment of the disclosure herein and having an absence of peaks at either: 3.0 to 4.2 degrees 2-theta±0.2 degrees 2-theta or 3.5 to 4.0 degrees 2-theta±0.2 degrees 2-theta.

In any aspect or embodiment of the present disclosure, crystalline Form 1 of Fosgonimeton is isolated. Particularly, crystalline Form 1 of Fosgonimeton according to any aspect or embodiment of the disclosure may be isolated.

In any aspect or embodiment crystalline Form 1 of Fosgonimeton may be polymorphically pure.

In any aspect or embodiment of the present disclosure, crystalline Form 1 of Fosgonimeton may be anhydrous.

Crystalline Form 1 of Fosgonimeton may be characterized by each of the above characteristics alone/or by all possible combinations, e.g., an XRPD pattern having peaks at 5.2, 9.4, 12.9, 13.7 and 18.9 degrees 2-theta±0.2 degrees 2-theta; an XRPD pattern as depicted in FIG. 1, and combinations thereof.

The present disclosure further provides a process for preparing crystalline Fosgonimeton Form 1 as defined in any aspect or embodiment herein, which comprises crystallisation of Fosgonimeton in a solvent mixture comprising water and at least one organic solvent selected from: an alcohol (preferably a C₁-C₆ alcohol, more preferably a C₁-C₃ alcohol, and most preferably ethanol or methanol), an ether (preferably a C₄-C₈ ether, more preferably a C₄-C₆ ether, and most preferably tetrahydrofuran), or acetonitrile.

The process may comprise crystallisation of Fosgonimeton from a solvent mixture selected from: ethanol and water, acetonitrile and water, methanol and water, or tetrahydrofuran and water. According to any aspect or embodiment, the ratio of organic solvent to water may be: about 3:1 to about 1:3, about 2.5:1 to about 1:2.5, about 2:1 to about 1:2, about 1.5:1 to about 1:1.5, about 1.2:1 to about 1:1.2, about 1.1:1 to about 1:1.1, or about 1:1. According to any aspect or embodiment of the process, the organic solvent may be used in an amount of: about 10 ml to about 100 ml, about 10 ml to about 80 ml, about 10 ml to about 70 ml, about 15 ml to about 65 ml, about 20 ml to about 60 ml, or about 20 ml to about 55 ml, or about 25 to about 50 ml per gram of Fosgonimeton.

According to any aspect or embodiment the process for preparing Fosgonimeton may comprise:
(i) providing a solution of Fosgonimeton in the solvent mixture;
(ii) optionally cooling, and optionally stirring;
(iii) optionally isolating; and
(iv) optionally drying the crystalline Fosgonimeton.

According to any aspect or embodiment, the solvent mixture in step (i) may comprise: water and methanol, water and isopropanol, or water and acetonitrile, preferably wherein the ratio of water to the organic solvent is about 1:1.

Optionally, step (i) may be carried out at a temperature of: about 45° C. to about 100° C., about 50° C. to about 90° C., about 55° C. to about 80° C., about 60° C. to about 80° C., or about 68° C. to about 77° C.

According to any aspect or embodiment, the cooling in step (ii) may carried out, preferably wherein the mixture is cooled to a temperature of: about −20° C. to about 30° C., about −18° C. to about 28° C., about −16° C. to about 25° C., or about −15° C. to about 25° C.

According to an aspect or embodiment of the process, the stirring in step (ii) may be carried out for period of: about 5 minutes to about 10 hours, about 10 minutes to about 6 hours, about 10 minutes to about 4 hours, about 10 minutes to about 2 hours, or about 10 minutes to about 1 hour.

According to any aspect or embodiment, step (iii) may be carried out, preferably by filtration, decantation or centrifuge, more preferably by filtration.

The process may further comprise combining the Fosgonimeton with at least one pharmaceutically acceptable excipient to form a pharmaceutical composition.

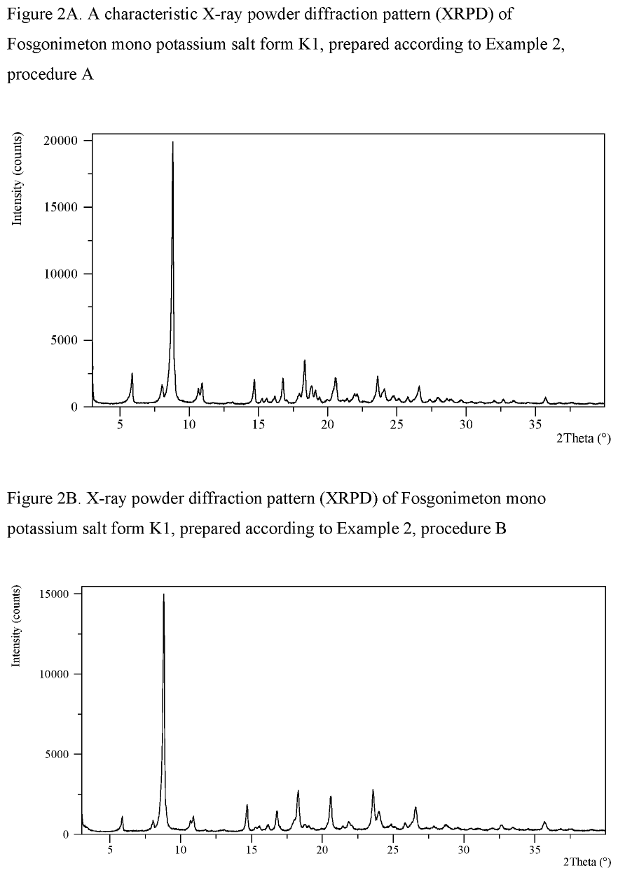

The present disclosure includes a crystalline polymorph of Fosgonimeton mono potassium salt designated Form K1. The crystalline Form K1 of Fosgonimeton mono potassium salt may be characterized by data selected from one or more of the following: an X-ray powder diffraction pattern substantially as depicted in FIG. 2A or 2B; an X-ray powder diffraction pattern having peaks at 5.9, 8.8 and 23.6 degrees 2-theta±0.2 degrees 2-theta; and combinations of these data.

Crystalline Form K1 of Fosgonimeton mono potassium salt may be further characterized by an X-ray powder diffraction pattern having peaks at 5.9, 8.8 and 23.6 degrees 2-theta±0.2 degrees 2-theta, and also having any one or both additional peaks selected from 18.3 and 20.6 degrees 2-theta±0.2 degrees 2-theta.

The crystalline Form K1 of Fosgonimeton mono potassium salt may be alternatively characterized by an X-ray powder diffraction pattern having peaks at 5.9, 8.8, 18.3, 20.6 and 23.6±0.2 degrees 2-theta.

Crystalline Form K1 of Fosgonimeton mono potassium salt may be further characterized by an X-ray powder diffraction pattern having peaks at 5.9, 8.8, 18.3, 20.6 and 23.6±0.2 degrees 2-theta±0.2 degrees 2-theta, and also having any one, two, three, four or five additional peaks selected from 10.9, 14.7, 15.6, 16.8 and 26.6±0.2 degrees 2-theta±0.2 degrees 2-theta.

Crystalline Form K1 of Fosgonimeton mono potassium salt may be alternatively characterized by an X-ray powder diffraction pattern having peaks at 5.9, 8.8, 10.9, 14.7, 15.6, 16.8, 18.3, 20.6, 23.6, and 26.6 degrees 2-theta±0.2 degrees 2-theta.

According to any aspect or embodiment of the disclosure crystalline Fosgonimeton mono potassium salt Form K1 may be additionally characterized by an X-ray powder diffraction pattern having an absence of peaks at one or more of the following: 4.2 to 5.2 degrees 2-theta±0.2 degrees 2-theta; 6.5 to 8.0 degrees 2-theta±0.2 degrees 2-theta; and 9.4 to 10.2 degrees 2-theta±0.2 degrees 2-theta.

In any aspect or embodiment of the present disclosure, crystalline Form K1 of Fosgonimeton mono potassium salt is isolated. Particularly, crystalline Form K1 of Fosgonimeton mono potassium salt according to any aspect or embodiment of the disclosure may be isolated.

In any aspect or embodiment crystalline Form K1 of Fosgonimeton mono potassium salt may be polymorphically pure.

In any aspect or embodiment of the present disclosure, crystalline Form K1 of Fosgonimeton mono potassium salt may be anhydrous.

Crystalline Form K1 of Fosgonimeton mono potassium salt may be characterized by each of the above characteristics alone/or by all possible combinations, e.g., an XRPD pattern having peaks at 5.9, 8.8, 18.3, 20.6 and 23.6 degrees 2-theta±0.2 degrees 2-theta; an XRPD pattern as depicted in FIG. 2A or 2B, and combinations thereof.

The present disclosure further includes a process for preparing Fosgonimeton potassium salt Form K1 as described in any aspect or embodiment herein, which comprises:
(i) suspending Fosgonimeton mono potassium salt in a solvent mixture comprising water and an organic solvent selected from: an alcohol (preferably a C₁-C₆ alcohol, more preferably a C₁-C₃ alcohol, and most preferably methanol or isopropanol) or acetonitrile;
(ii) optionally stirring the suspension; and
(iii) optionally isolating the crystalline Fosgonimeton.

According to any aspect or embodiment of the process, the organic solvent may be used in an amount of: about 4 ml to about 18 ml, about 5 ml to about 17 ml, about 6 ml to about 16 ml, about 7 ml to about 15 ml, about 8 ml to about 14 ml, or about 9 ml to about 13.5 ml, or about 9.4 or about 13.1 ml, per gram of Fosgonimeton mono potassium salt.

According to any aspect or embodiment of the process, the solvent mixture in step (i) may comprise: water and methanol, water and isopropanol, or water and acetonitrile.

Preferably, according to any aspect or embodiment, water may be used in an amount of: about 8% to about 20%, about 9% to about 18%, about 10% to about 17%, about 11% to about 16%, or about 11.8%, about 14.6%, or about 15.8%, by volume relative to the solvent mixture.

According to any aspect or embodiment of the process, step (i) may be carried out at a temperature of: about 18° C. to about 30° C., about 20° C. to about 30° C., about 22° C. to about 27° C., about 20° C. to about 25° C., or about 25° C.

According to any aspect or embodiment of the process, step (ii) is preferably carried out, preferably at a temperature of: about 18° C. to about 30° C., about 20° C. to about 30° C., about 22° C. to about 27° C., about 20° C. to about 25° C., or about 25° C.

According to any aspect or embodiment of the process, the suspension of Fosgonimeton potassium salt in step (i) may be prepared by a process comprising:
(a) providing a suspension of Fosgonimeton in the organic solvent to form a suspension; and
(b) combining the suspension with an aqueous solution of a base selected from potassium carbonate, potassium hydrogen carbonate, or potassium hydroxide, preferably potassium hydroxide.

According to any aspect or embodiment, the process step (b) may comprise addition of the aqueous solution of a base to the suspension of Fosgonimeton in step (a), preferably wherein the addition is dropwise. Preferably, according to any aspect or embodiment of the process, the base may be potassium hydroxide, and preferably wherein the base is used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton.

According to any aspect or embodiment of the process, step (b) may be carried out at a temperature of: about 18° C. to about 30° C., about 20° C. to about 30° C., about 22° C. to about 27° C., about 20° C. to about 25° C., or about 25° C. Optionally, step (b) may be carried out for a period of: about 1 hour to about 12 days, about 2 hours to about 10 days, about 3 hours to about 8 days, about 3 hours to about 7 days.

According to any aspect or embodiment of the process, the Fosgonimeton in step (a) may be Form 1 as defined in any aspect or embodiment of the disclosure herein.

According to any aspect or embodiment of the process, step (iii) is preferably carried out, preferably by filtration, decantation or centrifuge, more preferably by filtration. The process may further comprise combining the Fosgonimeton mono potassium salt with at least one pharmaceutically acceptable excipient to form a pharmaceutical composition.

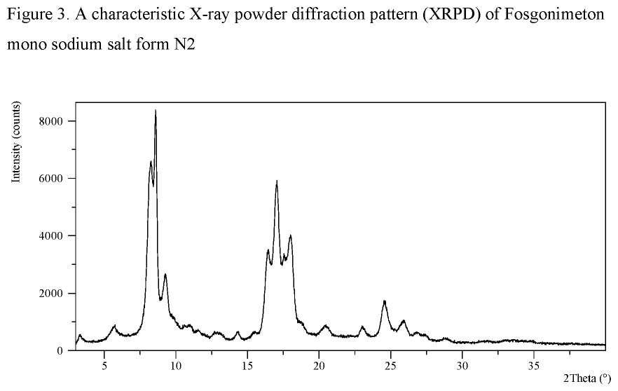

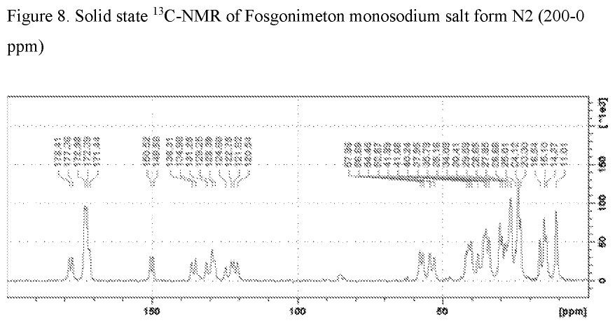

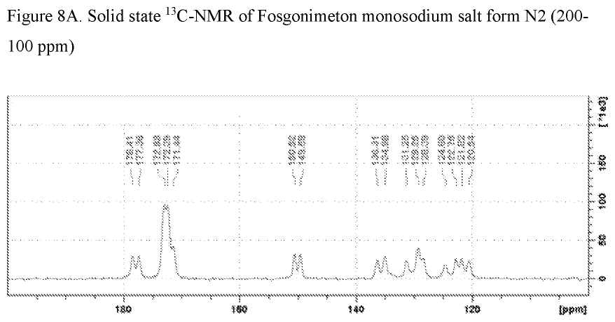

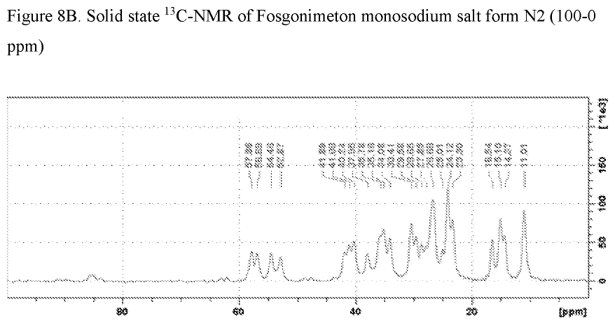

The present disclosure includes Fosgonimeton sodium salt, particularly Fosgonimeton mono sodium salt, and especially crystalline Fosgonimeton mono sodium salt. The present disclosure includes a crystalline polymorph of Fosgonimeton mono sodium salt designated Form N2. The crystalline Form N2 of Fosgonimeton mono sodium salt may be characterized by data selected from one or more of the following: an X-ray powder diffraction pattern substantially as depicted in FIG. 3; an X-ray powder diffraction pattern having peaks at 8.6, 9.3 and 16.4 degrees 2-theta±0.2 degrees 2-theta; a solid state ¹³C NMR spectrum having characteristic peaks at 178.4, 136.3, 54.5, 24.1 and 16.5 ppm±0.2 ppm; A solid state ¹³C NMR spectrum having the following chemical shift absolute differences from reference peak at 16.5 ppm±0.2 ppm: 167.4, 125.3, 43.5, 13.1 and 5.5 ppm±0.1 ppm; a solid state ¹³C NMR spectrum substantially as depicted in FIG. 8, 8A or 8B; and combinations of these data.

Crystalline Form N2 of Fosgonimeton mono sodium salt may be further characterized by an X-ray powder diffraction pattern having peaks at 8.6, 9.3 and 16.4 degrees 2-theta±0.2 degrees 2-theta, and also having any one or both additional peaks selected from 17.1 and 18.1 degrees 2-theta±0.2 degrees 2-theta.

The crystalline Form N2 of Fosgonimeton mono sodium salt may be alternatively characterized by an X-ray powder diffraction pattern having peaks at 8.6, 9.3, 16.4, 17.1 and 18.1 degrees 2-theta±0.2 degrees 2-theta.

Crystalline Form N2 of Fosgonimeton mono sodium salt may be further characterized by an X-ray powder diffraction pattern having peaks at 8.6, 9.3, 16.4, 17.1 and 18.1 degrees 2-theta±0.2 degrees 2-theta, and also having any one, two, three, four or five additional peaks selected from 5.7, 14.3, 23.0, 24.6 and 26.0±0.2 degrees 2-theta±0.2 degrees 2-theta.

Crystalline Form N2 of Fosgonimeton mono sodium salt may be alternatively characterized by an X-ray powder diffraction pattern having peaks at 5.7, 8.6, 9.3, 14.3, 16.4, 17.1, 18.1, 23.0, 24.6 and 26.0 degrees 2-theta±0.2 degrees 2-theta.

According to any aspect or embodiment of the disclosure crystalline Fosgonimeton mono sodium salt Form N2 may be additionally characterized by an X-ray powder diffraction pattern having an absence of peaks at one or more of the following: 4.5 to 5.0 degrees 2-theta±0.2 degrees 2-theta; 6.5 to 7.2 degrees 2-theta±0.2 degrees 2-theta; 13.6 to 13.8 degrees 2-theta±0.2 degrees 2-theta; and 14.8 to 15.0 degrees 2-theta±0.2 degrees 2-theta.

In any aspect or embodiment of the present disclosure, crystalline Form N2 of Fosgonimeton mono sodium salt is isolated. Particularly, crystalline Form N2 of Fosgonimeton mono sodium salt according to any aspect or embodiment of the disclosure may be isolated.

In any aspect or embodiment crystalline Form N2 of Fosgonimeton mono sodium salt may be polymorphically pure.

In any aspect or embodiment of the present disclosure, crystalline Form N2 of Fosgonimeton mono sodium salt may be hydrated, preferably form N2 is a monohydrate. In any embodiment, crystalline Form N2 of Fosgonimeton monosodium salt may contain from about 2% to about 4% of water, preferably about 3%, particularly about 3.2% of water, as measured by TGA.

Crystalline Form N2 of Fosgonimeton mono sodium salt may be characterized by each of the above characteristics alone/or by all possible combinations, e.g., an XRPD pattern having peaks at 8.6, 9.3 and 16.4 degrees 2-theta±0.2 degrees 2-theta; an XRPD pattern as depicted in FIG. 3, and combinations thereof.

Crystalline Form N2 of Fosgonimeton mono sodium salt according to any aspect or embodiment of the present disclosure may be prepared by a process comprising:
(i) suspending Fosgonimeton mono sodium salt in a solvent mixture comprising water and an organic solvent which is an alcohol (preferably a C₁-C₆ alcohol, more preferably a C₁-C₃ alcohol, and most preferably methanol or isopropanol) wherein the alcohol is used in an amount of: about 4 ml to about 12 ml, about 6 ml to about 11 ml, about 7 ml to about 10 ml, per gram of Fosgonimeton mono sodium salt; or suspending Fosgonimeton mono sodium salt in a solvent mixture comprising water and an organic solvent selected from: an ether (preferably a C₄-C₈ ether, more preferably a C₄-C₆ ether, particularly tetrahydrofuran or 2-methyltetrahydrofuran and most preferably 2-methyltetrahydrofuran), or an ester (preferably a C₄-C₈ ester, more preferably a C₄-C₆ ester, and particularly ethyl acetate), preferably wherein the ether or ester is used in an amount of: about 4 ml to about 20 ml, about 5 ml to about 15 ml, about 6 ml to about 11 ml, about 7 ml to about 10 ml, about 8 ml to about 10 ml, or about 9 ml to about 10 ml, or about 9.7 ml or about 10 ml per gram of Fosgonimeton mono sodium salt;
(ii) optionally stirring the suspension; and
(iii) optionally isolating the crystalline Fosgonimeton.

According to any embodiment of the process, the solvent mixture in step (i) may comprise: water and methanol, water and isopropanol, water and 2-methyltetrahydrofuran, or water and ethyl acetate. In any aspect or embodiment water may be used in an amount of: about 8% to about 15%, about 8% to about 13%, about 8% to about 12%, about 9% to about 11%, or about 10%, by volume relative to the solvent mixture. Step (i) may be carried out at a temperature of: about 18° C. to about 30° C., about 20° C. to about 30° C., about 22° C. to about 27° C., about 20° C. to about 25° C., or about 25° C. According to any embodiment of the process, step (ii) is carried out, preferably at a temperature of: about 18° C. to about 30° C., about 20° C. to about 30° C., about 22° C. to about 27° C., about 20° C. to about 25° C., or about 25° C.

According to any aspect or embodiment of the process, the suspension of Fosgonimeton sodium salt in step (i) may be prepared by a process comprising:
(a) providing a suspension of Fosgonimeton in the organic solvent to form a suspension; and
(b) combining the suspension with an aqueous solution of a base selected from sodium carbonate, sodium hydrogen carbonate, or sodium hydroxide, preferably sodium carbonate or sodium hydroxide.

According to any aspect or embodiment of the process, step (b) may comprise addition of the aqueous solution of a base to the suspension of Fosgonimeton in step (a), preferably wherein the addition is dropwise. Optionally, the base may be sodium hydrogen carbonate or sodium hydroxide, and the base may be used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton. Alternatively, the base may be sodium carbonate, and the base may be used in an amount of: about 0.4 to about 4, about 0.5 to about 2, about 0.5 to about 1.5, about 0.5 to about 1.0, about 0.5 to about 0.8; or about 0.5 to about 0.6 mole equivalents relative to Fosgonimeton.

According to any aspect or embodiment of the process, step (b) may be carried out at a temperature of: about 18° C. to about 30° C., about 20° C. to about 30° C., about 22° C. to about 27° C., about 20° C. to about 25° C., or about 25° C. Optionally, step (b) may be carried out for a period of: about 1 hour to about 12 days, about 2 hours to about 10 days, about 3 hours to about 8 days, about 4 hours to about 7 days.

The starting material for the process for preparing Fosgonimeton mono sodium salt Form N2 may preferably be Form 1 of Fosgonimeton as described in any aspect or embodiment of the disclosure herein.

According to any aspect or embodiment of the process, step (iii) is preferably carried out, more preferably by filtration, decantation or centrifuge, most preferably by filtration.

The process for preparing Form N2 of Fosgonimeton mono sodium according to any aspect or embodiment of the disclosure may further comprise combining the Fosgonimeton mono sodium salt with at least one pharmaceutically acceptable excipient to form a pharmaceutical composition.

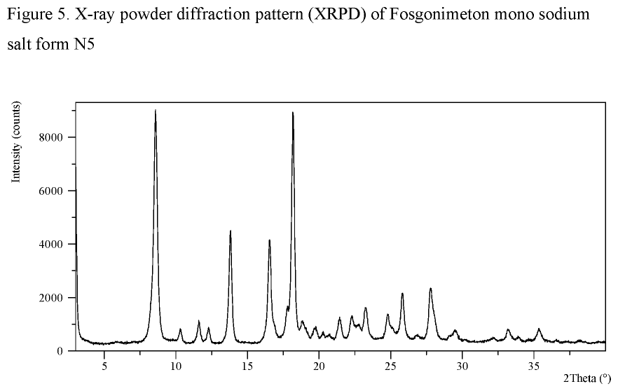

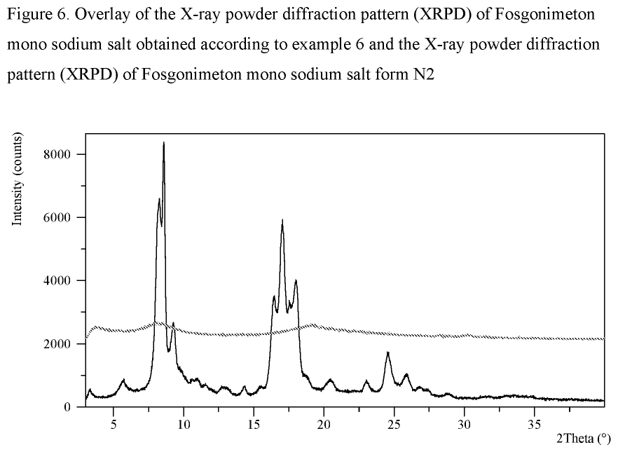

The present disclosure includes a crystalline polymorph of Fosgonimeton mono sodium salt designated Form N5. The crystalline Form N5 of Fosgonimeton mono sodium salt may be characterized by an X-ray powder diffraction pattern substantially as depicted in FIG. 5; an X-ray powder diffraction pattern having peaks at 13.8, 21.4 and 27.7 degrees 2-theta±0.2 degrees 2-theta.

Crystalline Form N5 of Fosgonimeton mono sodium salt may be further characterized by an X-ray powder diffraction pattern having peaks at 13.8, 21.4 and 27.7 degrees 2-theta±0.2 degrees 2-theta, and also having any one or both additional peaks selected from 10.3 and 19.6 degrees 2-theta±0.2 degrees 2-theta.

The crystalline Form N5 of Fosgonimeton mono sodium salt may be alternatively characterized by an X-ray powder diffraction pattern having peaks at 10.3, 13.8, 19.6, 21.4, and 27.7 degrees 2-theta±0.2 degrees 2-theta.

Crystalline Form N5 of Fosgonimeton mono sodium salt may be further characterized by an X-ray powder diffraction pattern having peaks at 10.3, 13.8, 19.6, 21.4, and 27.7 degrees 2-theta±0.2 degrees 2-theta, and also having any one, two or three additional peaks selected from 11.6, 12.3 and 24.8±0.2 degrees 2-theta±0.2 degrees 2-theta.

Crystalline Form N5 of Fosgonimeton mono sodium salt may be alternatively characterized by an X-ray powder diffraction pattern having peaks at 10.3, 11.6, 12.3, 13.8, 19.6, 21.4, 24.8 and 27.7 degrees 2-theta±0.2 degrees 2-theta.

According to any aspect or embodiment of the disclosure crystalline Fosgonimeton mono sodium salt Form N5 may be additionally characterized by an X-ray powder diffraction pattern having an absence of peaks at one or more of the following: 3.6 to 5.4 degrees 2-theta±0.2 degrees 2-theta; 9.2 to 9.8 degrees 2-theta±0.2 degrees 2-theta; 10.7 to 11.0 degrees 2-theta±0.2 degrees 2-theta; 12.8 to 13.2 degrees 2-theta±0.2 degrees 2-theta; 14.4 to 16.0 degrees 2-theta±0.2 degrees 2-theta; and 30.2 to 31.6 degrees 2-theta±0.2 degrees 2-theta.

In any aspect or embodiment of the present disclosure, crystalline Form N5 of Fosgonimeton mono sodium salt is isolated. Particularly, crystalline Form N5 of Fosgonimeton mono sodium salt according to any aspect or embodiment of the disclosure may be isolated.

In any aspect or embodiment crystalline Form N5 of Fosgonimeton mono sodium salt may be polymorphically pure.

In any aspect or embodiment of the present disclosure, crystalline Form N5 of Fosgonimeton mono sodium salt Form N5 as described in any may be hydrated, preferably form N5 is a monohydrate. In any embodiment, crystalline Form N5 of Fosgonimeton monosodium salt may contain from about 2% to about 4% of water, preferably about 3%, particularly about 3.2% of water, as measured by TGA.

Crystalline Form N5 of Fosgonimeton mono sodium salt may be characterized by each of the above characteristics alone/or by all possible combinations, e.g., an XRPD pattern having peaks at 13.8, 21.4 and 27.7 degrees 2-theta±0.2 degrees 2-theta; an XRPD pattern as depicted in FIG. 5, and combinations thereof.

The present disclosure further provides a process for preparing Fosgonimeton mono sodium salt Form N5 as described in any aspect or embodiment herein, which comprises:
(i) suspending Fosgonimeton mono sodium salt in a solvent mixture comprising water and an organic solvent which is an alcohol (preferably a C₁-C₆ alcohol, more preferably a C₁-C₃ alcohol, and most preferably methanol) wherein the alcohol is used in an amount of: about 15 ml to about 30 ml, about 15 ml to about 28 ml, about 16 ml to about 25 ml, about 16 ml to about 22 ml, about 17 ml to about 21 ml, about 18 ml to about 20 ml, or about 19 or 19.2 ml, per gram of Fosgonimeton mono sodium salt;
(ii) optionally stirring the suspension; and
(iii) optionally isolating the crystalline Fosgonimeton.

According to any aspect or embodiment of the process for preparing Fosgonimeton mono sodium salt Form N5, the solvent mixture in step (i) may comprise water and an alcohol, preferably water and methanol. Water may be used in an amount of: about 2% to about 7%, about 2% to about 6%, about 3% to about 6%, about 3% to about 5%, or about 4%, by volume relative to the solvent mixture. In any aspect or embodiment of the process, step (i) may be carried out at a temperature of: about 18° C. to about 30° C., about 20° C. to about 30° C., about 22° C. to about 27° C., about 20° C. to about 25° C., or about 25° C.

In any aspect or embodiment of the process for preparing Fosgonimeton mono sodium salt Form N5, step (ii) is preferably carried out, preferably at a temperature of: about 18° C. to about 30° C., about 20° C. to about 30° C., about 22° C. to about 27° C., about 20° C. to about 25° C., or about 25° C.

In any aspect or embodiment of the process, wherein the suspension of Fosgonimeton sodium salt in step (i) is prepared by a process comprising:
(a) providing a suspension of Fosgonimeton in the organic solvent to form a suspension; and
(b) combining the suspension with an aqueous solution of a base selected from sodium carbonate, sodium hydrogen carbonate, or sodium hydroxide, preferably sodium carbonate or sodium hydroxide.

According to any aspect or embodiment of the process, step (b) may comprise addition of the aqueous solution of a base to the suspension of Fosgonimeton in step (a), preferably wherein the addition is dropwise. Preferably, the base is sodium hydrogen carbonate or sodium hydroxide, and wherein the base is used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton. More preferably, the base is sodium carbonate, wherein the base is used in an amount of: about 1 to about 4, about 1 to about 3, or about 1 to about 2, mole equivalents relative to Fosgonimeton.

Preferably, according to any aspect or embodiment of the process for preparing Fosgonimeton mono sodium salt Form N5, step (b) may be carried out at a temperature of: about 18° C. to about 30° C., about 20° C. to about 30° C., about 22° C. to about 27° C., about 20° C. to about 25° C., or about 25° C. Optionally, step (b) may be carried out for a period of: about 4 hours to about 7 days, about 8 hours to about 5 days, about 12 hours to about 4 days, about 18 hours to about 3 days, or about 1 to about 2 days.

Preferably, according to any aspect embodiment of the process for preparing Fosgonimeton mono sodium salt Form N5, the Fosgonimeton in step (a) is Form 1 as defined in any aspect or embodiment of the disclosure herein.

According to any aspect or embodiment of this process, step (iii) may be carried out, preferably by filtration, decantation or centrifuge, more preferably by filtration. The process may further comprise combining the Fosgonimeton mono sodium salt with at least one pharmaceutically acceptable excipient to form a pharmaceutical composition.

The present disclosure further encompasses Fosgonimeton Cysteine designated form C1. In embodiments the molar ratio of Fosgonimeton to cysteine [n(Fosgonimeton):n(Cysteine)] in form C1 is typically about 1:2.

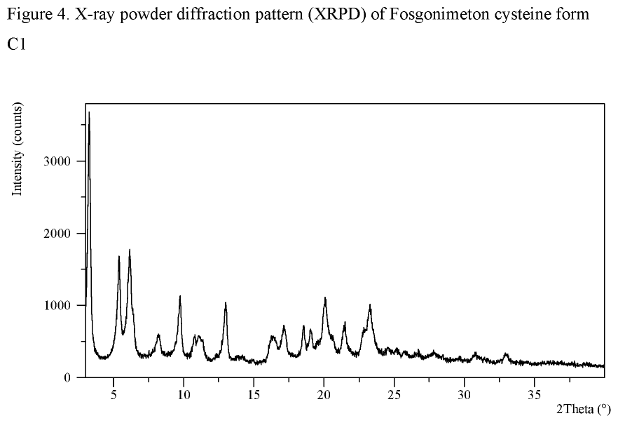

Crystalline Form C1 of Fosgonimeton cysteine may be characterized by data selected from one or more of the following: an X-ray powder diffraction pattern substantially as depicted in FIG. 4; an X-ray powder diffraction pattern having peaks at 6.1, 9.7 and 20.1 degrees 2-theta±0.2 degrees 2-theta; and combinations of these data.

Crystalline Form C1 of Fosgonimeton cysteine may be further characterized by an X-ray powder diffraction pattern having peaks at 6.1, 9.7 and 20.1 degrees 2-theta±0.2 degrees 2-theta, and also having any one or both additional peaks selected from 8.2 and 17.2 degrees 2-theta±0.2 degrees 2-theta.

The crystalline Form C1 of Fosgonimeton cysteine may be alternatively characterized by an X-ray powder diffraction pattern having peaks at 6.1, 8.2, 9.7, 17.2 and 20.1 degrees 2-theta±0.2 degrees 2-theta.

Crystalline Form C1 of Fosgonimeton cysteine may be further characterized by an X-ray powder diffraction pattern having peaks at 6.1, 8.2, 9.7, 17.2 and 20.1±0.2 degrees 2-theta±0.2 degrees 2-theta, and also having any one, two, three, four or five additional peaks selected from 3.3, 5.4, 13.1, 21.5 and 23.3±0.2 degrees 2-theta±0.2 degrees 2-theta.

Crystalline Form C1 of Fosgonimeton cysteine may be alternatively characterized by an X-ray powder diffraction pattern having peaks at 3.3, 5.4, 6.1, 8.2, 9.7, 13.1, 17.2, 20.1, 21.5 and 23.3 degrees 2-theta±0.2 degrees 2-theta.

In any aspect or embodiment of the present disclosure, crystalline Form C1 of Fosgonimeton cysteine is isolated. Particularly, crystalline Form C1 of Fosgonimeton cysteine according to any aspect or embodiment of the disclosure may be isolated.

In any aspect or embodiment crystalline Form C1 of Fosgonimeton cysteine may be polymorphically pure.

Crystalline Form C1 of Fosgonimeton cysteine may be characterized by each of the above characteristics alone/or by all possible combinations, e.g., an XRPD pattern having peaks at 6.1, 9.7 and 20.1 degrees 2-theta±0.2 degrees 2-theta; an XRPD pattern as depicted in FIG. 4, and combinations thereof.

The present disclosure encompasses a process for preparing other solid state forms of Fosgonimeton, Fosgonimeton salts and solid state forms, or cocrystals thereof. The process includes preparing any one of the solid state form of Fosgonimeton or the salts thereof by the processes of the present disclosure, and converting that form to a different form of Fosgonimeton or the salts, or cocrystals thereof. For example, the process may include preparing any one of the solid state form of Fosgonimeton potassium of the present disclosure by the processes of the present disclosure, and converting it to said other form of Fosgonimeton or Fosgonimeton salt or cocrystal. The conversion can be done, for example, by a process comprising acidifying the described form of Fosgonimeton potassium and reacting the obtained Fosgonimeton with an appropriate base, to obtain the corresponding salt.

The present disclosure provides the above described crystalline polymorphs of Fosgonimeton or the salt thereof for use in the preparation of pharmaceutical compositions comprising Fosgonimeton or the salt thereof and/or solid state forms thereof.

The present disclosure also encompasses the use of crystalline polymorphs of Fosgonimeton or the salt thereof of the present disclosure for the preparation of pharmaceutical compositions of crystalline polymorph of Fosgonimeton or the salt thereof and/or solid state forms thereof.

The present disclosure includes processes for preparing the above mentioned pharmaceutical compositions. The processes include combining any one or a combination of the crystalline polymorph of Fosgonimeton or the salt thereof of the present disclosure with at least one pharmaceutically acceptable excipient.

Pharmaceutical combinations or formulations of the present disclosure contain any one or a combination of the solid state form of Fosgonimeton or the salt thereof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Fosgonimeton and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Fosgonimeton can be administered. Fosgonimeton may be formulated for administration to a mammal, in embodiments to a human, by injection. Fosgonimeton can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Fosgonimeton and the pharmaceutical compositions and/or formulations of Fosgonimeton of the present disclosure can be used as medicaments, particularly for the treatment of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and schizophrenia, and in embodiments for the treatment of patients with Diabetic Peripheral Neuropathic pain and/or Post-Herpetic Neuralgia.

The present disclosure also provides methods of treating of patients with Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and schizophrenia, and more particularly Diabetic Peripheral Neuropathic pain and/or Post-Herpetic Neuralgia by administering a therapeutically effective amount of any one or a combination of the crystalline polymorphs of Fosgonimeton of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample is applied directly on a silicon plate holder. The X-ray powder diffraction pattern was measured with Philips X′Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan.

### SS-NMR Method

The spectra were recorded using BRUKER AVANCE III HD US/WB spectrometer at 11.7 T with 3.2 mm probe head. The spectra were recorded at room temperature and the frictional heating of the spinning samples was compensated.

The ¹³C CP/MAS NMR spectra employing cross-polarization were acquired using the standard cross-polarization pulse scheme at spinning frequency of 15 kHz. The duration of cross-polarization spin-lock pulse was 2 ms, and the spectral width was 300 ppm with the resonance frequency offset 100 ppm.

As a dipolar decoupling SPINAL64 pulse sequence was used. The number of scans is set for the signal-to-noise ratio SINO reaches at least the value ca. 50. The ¹³C scales are referenced to glycine (176.03 ppm).

The NMR spectrometer was calibrated and all experimental parameters are carefully optimized prior the recording of the spectra. Magic angle is set using KBr during the standard optimization procedure and homogeneity of magnetic field is optimized using adamantane sample (resulting line-width at half-height Δv½ was less than 3.5 Hz at 250 ms of acquisition time).

### EXAMPLES

### Preparation of Starting Materials

Fosgonimeton can be prepared according to methods known from the literature, for example according to the disclosure in International Publication No. WO 2017/210489.

### Example 1: Preparation of Fosgonimeton Crystal Form 1

### Procedure A

1.0 gram of Fosgonimeton was dissolved in 50 mL of EtOH:Water (1:1) at 68° C. Solution was cooled down to room temperature while stirred. Crystallization occurred during cooling at about 29° C. Suspension was filtered off and obtained solid was dried in vacuum oven at 70° C. for 3 hours. Sample was analyzed by XRPD and the XRPD pattern is presented in FIG. 1.

### Procedure B

1.0 gram of crude Fosgonimeton was dissolved in 100 mL of ACN:Water (1:1) mixture of solvents at 77° C. Solution was cooled down to 10° C. Crystallization occurred after a while. Suspension was filtered off and obtained solid was dried in vacuum oven at 70° C. for 3 hours. The sample was analyzed by XRPD and identified as Fosgonimeton form 1.

### Procedure C

100 mg of crude Fosgonimeton was dissolved in 10 mL of MeOH:Water (1:1) mixture of solvents at 74° C. Heating was discontinued. Crystallization occurred at 45° C. Suspension was filtered off and obtained solid was dried in vacuum oven at 80° C. for 3 hours. The sample was analyzed by XRPD and identified as Fosgonimeton form 1.

### Procedure D

100 mg of crude Fosgonimeton was dissolved in 10 mL of THF:Water (1:1) mixture of solvents at 74° C. Solution was cooled down to −15° C. Crystallization occurred at −10° C. Suspension was filtered off and obtained solid was dried in vacuum oven at 80° C. for 3 hours. The sample was analyzed by XRPD and identified as Fosgonimeton form 1.

### Example 2: Preparation of Fosgonimeton Mono Potassium Salt Crystal Form K1

### Procedure A

500 mg (1 eq) of Fosgonimeton was suspended in 7 mL of MeOH at room temperature. 940.7 μL (1.1 eq) of 1 M KOH solution was added dropwise. Suspension was stirred for 4 hours at room temperature and filtered off Sample was analyzed by XRPD and the XRPD pattern is presented in FIG. 2A.

### Procedure B

100 mg (1 eq.) of Fosgonimeton form 1 (prepared according to Example 1) was suspended in 1.0 mL of MeOH at room temperature. 188.1 μL (1.1 eq) of 1M KOH solution was added dropwise. Suspension was stirred for 3 hours at room temperature and filtered off. The sample was analyzed by XRPD and identified as Fosgonimeton potassium form K1 and the XRPD pattern is presented in FIG. 2B.

### Procedure C

50 mg (1 eq.) of crude Fosgonimeton was suspended in 0.5 mL of MeOH at room temperature. 94.1 μL (1.1 eq) of 1M KOH solution was added dropwise. Suspension was stirred for 1 day at room temperature and filtered off. The sample was analyzed by XRPD and identified as Fosgonimeton mono potassium salt form K1.

### Procedure D

50 mg (1 eq.) of crude Fosgonimeton was suspended in 0.5 mL of 2-Propanol at room temperature. 94.1 μL (1.1 eq) of 1M KOH solution was added dropwise. Suspension was stirred for 7 days at room temperature and filtered off. The sample was analyzed by XRPD and identified as Fosgonimeton mono potassium salt form K1.

### Procedure E

50 mg (1 eq.) of crude Fosgonimeton was suspended in 0.5 mL of Acetonitrile at room temperature. 85.5 μL (1 eq) of 1M KOH solution was added dropwise. Suspension was stirred for 1 day at room temperature and filtered off. The sample was analyzed by XRPD and identified as Fosgonimeton mono potassium salt form K1.

### Example 3: Preparation of Fosgonimeton Mono Sodium Salt Crystal Form N2

### Procedure A

200 mg (2 eq.) of Fosgonimeton form 1 (prepared according to Example 1) was suspended in 1.6 mL of MeOH (c=125 g/L) at room temperature. 171 μL (1 eq) of 1M Na₂CO₃ solution was added dropwise. Suspension was stirred for 4 hours at room temperature and filtered off Sample was analyzed by XRPD and the XRPD pattern is presented in FIG. 3.

### Procedure B

50 mg (1 eq.) of crude Fosgonimeton was suspended in 0.5 mL of 2-Propanol at room temperature. 94.1 μL (1.1 eq) of 1M NaOH solution was added dropwise. Suspension was stirred for 1 day at room temperature and filtered off. The sample was analyzed by XRPD and identified as Fosgonimeton monosodium salt form N2.

### Procedure C

50 mg (2 eq.) of crude Fosgonimeton was suspended in 0.5 mL of 2-Methyl tetrahydrofuran at room temperature. 42.8 μL (1 eq) of 1M Na₂CO₃ solution was added dropwise. Suspension was stirred for 7 days at room temperature and filtered off. The sample was analyzed by XRPD and identified as Fosgonimeton monosodium salt form N2.

### Procedure D

50 mg (2 eq.) of crude Fosgonimeton was suspended in 0.5 mL of Ethyl acetate at room temperature. 42.8 μL (1 eq) of 1M Na₂CO₃ solution was added dropwise. Suspension was stirred for 7 days at room temperature and filtered off. The sample was analyzed by XRPD and identified as Fosgonimeton monosodium salt form N2.

### Example 4: Preparation of Fosgonimeton Cysteine Co-Crystal Form C1

982 mg (1 eq) of crude Fosgonimeton and 1018 mg of Cysteine (5 eq) was dissolved in 34 mL of a mixtures of solvents, 2-Propanol:water (1:1), at 65° C. Heating was discontinued. Solution crystallized at about 35° C. Obtained solid was filtered off Sample was analyzed by XRPD and the XRPD pattern is presented in FIG. 4.

### Example 5: Preparation of Fosgonimeton Mono Sodium Salt Crystal Form N5

### Procedure A

200 mg (1 eq.) of Fosgonimeton acid form 1 was suspended in 4.0 mL of Methanol (concentration 50 mg/mL) at room temperature. 171 μL (2 eq) of 1M Na₂CO₃ solution was added dropwise. Suspension was stirred for 1 day at room temperature and filtered off Sample was analyzed by XRPD and the XRPD pattern is presented in FIG. 5.

### Procedure B

1.0 gram (2 eq.) of crude Fosgonimeton was suspended in 20 mL of MeOH (c=50 g/L) at room temperature. 855.2 μL (1 eq) of 1M Na₂CO₃ solution was added dropwise. Suspension was stirred for 2 days at room temperature and filtered off Sample was analyzed by XRPD. Fosgonimeton monosodium salt, form N5 was obtained.

### Example 6: Preparation of Amorphous Fosgonimeton Mono Sodium Salt

150 mg of Fosgonimeton form N2 prepared according to Example 3, procedure - - - was milled in teflon jar with 7 teflon balls in Retsch mill 200 for 3 cycles of 90 minutes. Frequency was 25 Hz. Sample was analyzed by XRPD. Comparison with crystalline Fosgonimeton salt sodium salt form N2 is presented in FIG. 3.

### Example 7: Preparation of Amorphous Fosgonimeton

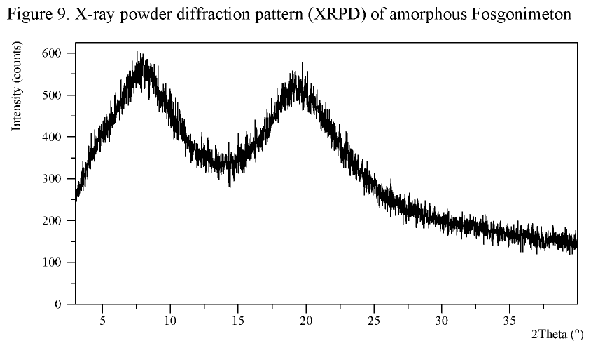

360 mg of starting material, Fosgonimeton acid form 1, was dry-milled in Retsch mill 200, in agate jar with 2 agate balls, at 25 Hz for 3 hours. Sample was analyzed by XRPD and the XRPD pattern is presented in FIG. 9.

### Example 8: Stability Studies

### Form N2 Fossgonimeton Mono Sodium Salt

Samples of Fosgonimeton mono sodium salt form N2 were subjected to conditions of different relative humidity (20%-100%) at ambient temperature. XRPD analysis was performed on the samples after 30 days. The results are shown in Table 1 below:

TABLE 1
Relative humidity (30 days at ambient temperature)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

Samples of Fosgonimeton mono sodium salt form N2 were subjected to slurry and to sauna conditions in various conditions. In the slurry experiments, 50 mg of the sample is placed in a 2 mL glass vial with 0.5 mL of solvent. Obtained suspension is stirred at ambient temperature for 1 or 7 days. XRPD analysis is performed on crystalline material after filtration. In the sauna experiments, 50 mg of crystalline material is placed in an Eppendorf tube. 0.5 mL of solvent is placed in a sealed 10 mL glass vial, along with Eppendorf tube. Crystalline material is checked by XRPD analysis after 1 or 7 days. XRPD analysis performed on each of the samples after filtration confirmed no change in the starting material (Table 2).

A sample of Fosgonimeton mono sodium salt form N2 was subjected to a pressure of 2 ton (Specac Hydraulic Press, set to 2 tons). XRPD analysis was performed on the samples after 2 minutes showed no polymorphic conversion.

A sample of Fosgonimeton mono sodium salt form N2 was subjected to heating to 100° C. for 60 minutes. XRPD analysis was performed on the samples after 60 minutes showed no polymorphic conversion.

Stability data for Form N2 Fosgonimeton mono sodium salt is summarized in Table 2 below:

TABLE 2

| Stress test | Parameters | XRPD |
|---|---|---|
| Heating | 100° C./60 minutes | No polymorphic change |
| Pressure | 2 tons/2 minutes | No polymorphic change |
| Slurry | Acetone/20-25° C./1 day | No polymorphic change |
| | Ethanol, 96%/20-25° C./1 day | No polymorphic change |
| | Ethyl acetate/20-25° C./1 day | No polymorphic change |
| | 2-Propanol/20-25° C./1 day | No polymorphic change |
| | Acetone/20-25° C./7 days | No polymorphic change |
| | Ethanol, 96%/20-25° C./7 days | No polymorphic change |
| | Water/20-25° C./7 days | No polymorphic change |
| Sauna | Acetone/20-25° C./7 days | No polymorphic change |
| | Ethanol, 96%/20-25° C./7 days | No polymorphic change |
| | Ethyl acetate/20-25° C./7 days | No polymorphic change |
| | 2-Propanol/20-25° C./7 days | No polymorphic change |
| | Water/20-25° C./7 days | No polymorphic change |

These results show that Fosgonimeton mono sodium crystalline form N2, has good storage stability, is resistant to polymorphic changes during heating, compression, slurry or sauna conditions, and is therefore highly suitable for preparing pharmaceutical formulations.

### Form N5 Fosgonimeton Mono Sodium Salt

Samples of Fosgonimeton mono sodium salt form N5 were subjected to conditions of different relative humidity (20%-100%) at ambient temperature. XRPD analysis was performed on the samples after 30 days. The results are shown in Table 3 below:

TABLE 3
Relative humidity (30 days at ambient temperature)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

Samples of Fosgonimeton mono sodium salt form N5 were subjected to slurry and to sauna conditions in various conditions. In the slurry experiments, 50 mg of the sample is placed in a 2 mL glass vial with 0.5 mL of solvent. Obtained suspension is stirred at ambient temperature for 1 or 7 days. XRPD analysis is performed on crystalline material after filtration. In the sauna experiments, 50 mg of crystalline material is placed in an Eppendorf tube. 0.5 mL of solvent is placed in a sealed 10 mL glass vial, along with Eppendorf tube. Crystalline material is checked by XRPD analysis after 1 or 7 days. XRPD analysis performed on each of the samples after filtration confirmed no change in the starting material (Table 4).

A sample of Fosgonimeton mono sodium salt form N5 was subjected to a pressure of 2 ton (Specac Hydraulic Press, set to 2 tons). XRPD analysis was performed on the samples after 2 minutes showed no polymorphic conversion.

A sample of Fosgonimeton mono sodium salt form N5 was subjected to heating to 100° C. for 60 minutes. XRPD analysis was performed on the samples after 60 minutes showed no polymorphic conversion.

Stability data for Form N2 Fosgonimeton mono sodium salt is summarized in Table 4 below:

TABLE 4

| Stress test | Parameters | XRPD |
|---|---|---|
| Pressure | 2 tons/2 minutes | No polymorphic change |
| Slurry | Acetone/20-25° C./7 days | No polymorphic change |
| | Ethyl acetate/20-25° C./1 day | No polymorphic change |
| | 2-Propanol/20-25° C./1 day | No polymorphic change |
| Sauna | Ethanol, 96%/20-25° C./7 days | No polymorphic change |
| | Ethyl acetate/20-25° C./7 days | No polymorphic change |
| | 2-Propanol/20-25° C./7 days | No polymorphic change |

These results show that Fosgonimeton mono sodium crystalline form N5, has good storage stability, is resistant to polymorphic changes under compression, slurry or Sauna conditions, and is therefore highly suitable for preparing pharmaceutical formulations.

### Form K1 Fossgonimeton Mono Potassium Salt

Samples of Fosgonimeton mono potassium salt form K1 were subjected to conditions of different relative humidity (20%-100%) at ambient temperature. XRPD analysis was performed on the samples after 30 days. The results are shown in Table 5 and Table 6 below:

TABLE 5
Relative humidity (30 days at ambient temperature)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

TABLE 6
Relative humidity (30 days at 40 ° C.)

| 75% | 100% |
|---|---|
| No polymorphic | No polymorphic |
| conversion | conversion |

Samples of Fosgonimeton mono potassium salt form K1 were subjected to slurry and to sauna conditions in various conditions. In the slurry experiments, 50 mg of the sample is placed in a 2 mL glass vial with 0.5 mL of solvent. Obtained suspension is stirred at ambient temperature for 1 or 7 days. XRPD analysis is performed on crystalline material after filtration. In the sauna experiments, 50 mg of crystalline material is placed in an Eppendorf tube. 0.5 mL of solvent is placed in a sealed 10 mL glass vial, along with Eppendorf tube. Crystalline material is checked by XRPD analysis after 1 or 7 days. XRPD analysis performed on each of the samples after filtration confirmed no change in the starting material (Table 7).

A sample of Fosgonimeton mono potassium salt form K1 was subjected to a pressure of 2 tons (Specac Hydraulic Press, set to 2 tons). XRPD analysis was performed on the samples after 2 minutes showed no polymorphic conversion.

A sample of Fosgonimeton mono potassium salt form K1 was subjected to heating to 100° C. for 60 minutes. XRPD analysis was performed on the samples after 60 minutes showed no polymorphic conversion.

Stability data for Form K1 Fosgonimeton mono potassium salt is summarized in Table 7 below:

TABLE 7

| Stress test | Parameters | XRPD |
|---|---|---|
| Heating | 100° C./60 minutes | No polymorphic change |
| Pressure | 2 tons/2 minutes | No polymorphic change |
| | Acetone/20-25° C./7 days | No polymorphic change |
| | Ethanol, 96%/20-25° C./7 days | No polymorphic change |
| | Ethyl acetate/20-25° C./7 days | No polymorphic change |
| | 2-Propanol/20-25° C./7 days | No polymorphic change |
| Sauna | Acetone/20-25° C./7 days | No polymorphic change |
| | Ethanol, 96%/20-25° C./7 days | No polymorphic change |
| | Ethyl acetate/20-25° C./7 days | No polymorphic change |
| | 2-Propanol/20-25° C./7 days | No polymorphic change |
| | Water/20-25° C./7 days | No polymorphic change |

These results show that Fosgonimeton mono potassium crystalline form K1, has good storage stability, is resistant to polymorphic changes during heating, compression, slurry or Sauna conditions, and is therefore highly suitable for preparing pharmaceutical formulations.

### Form 1 of Fossgonimeton

Samples of Fosgonimeton form 1 were subjected to conditions of different relative humidity (20%-100%) at ambient temperature. XRPD analysis was performed on the samples after 30 days. The results are shown in Table 8 below:

TABLE 8
Relative humidity (30 days at ambient temperature)

| 20% | 40% | 60% | 80% | 100% |
|---|---|---|---|---|
| No | No | No | No | No |
| polymorphic | polymorphic | polymorphic | polymorphic | polymorphic |
| conversion | conversion | conversion | conversion | conversion |

Samples of Fosgonimeton form 1 were subjected to slurry and to Sauna conditions in various conditions. In the slurry experiments, 50 mg of the sample is placed in a 2 mL glass vial with 0.5 mL of solvent. Obtained suspension is stirred at ambient temperature for 1 or 7 days. XRPD analysis is performed on crystalline material after filtration. In the sauna experiments, 50 mg of crystalline material is placed in an Eppendorf tube. 0.5 mL of solvent is placed in a sealed 10 mL glass vial, along with Eppendorf tube. Crystalline material is checked by XRPD analysis after 1 or 7 days. XRPD analysis performed on each of the samples after filtration confirmed no change in the starting material (Table 9).

A sample of Fosgonimeton form 1 was subjected to a pressure of 2 tons (Specac Hydraulic Press, set to 2 tons). XRPD analysis was performed on the samples after 2 minutes showed no polymorphic conversion.

A sample of Fosgonimeton form 1 was subjected to heating to 100° C. for 60 minutes. XRPD analysis was performed on the samples after 60 minutes showed no polymorphic conversion.

Stability data for Form N2 Fosgonimeton mono sodium salt is summarized in Table 9 below:

TABLE 9

| Stress test | Parameters | XRPD |
|---|---|---|
| Heating | 100° C./60 minutes | No polymorphic change |
| Pressure | 2 tons/2 minutes | No polymorphic change |
| Slurry | Acetone/20-25° C./1 day | No polymorphic change |
| | Ethanol, 96%/20-25° C./1 day | No polymorphic change |
| | Ethyl acetate/20-25° C./1 day | No polymorphic change |
| | 2-Propanol/20-25° C./1 day | No polymorphic change |
| | Acetone/20-25° C./7 days | No polymorphic change |
| Sauna | Ethanol, 96%/20-25° C./7 days | No polymorphic change |
| | Water/20-25° C./7 days | No polymorphic change |
| | Acetone/20-25° C./7 days | No polymorphic change |
| | Ethanol, 96%/20-25° C./7 days | No polymorphic change |
| | Ethyl acetate/20-25° C./7 days | No polymorphic change |

These results show that Fosgonimeton form 1, has good storage stability, is resistant to polymorphic changes during heating, compression, slurry or Sauna conditions, and is therefore highly suitable for preparing pharmaceutical formulations.

Further aspects and embodiments of the present disclosure are set out in the numbered clauses below:
1. Fosgonimeton sodium salt, preferably Fosgonimeton mono sodium salt.
2. Fosgonimeton sodium salt, preferably Fosgonimeton mono sodium salt according to Clause 1, which is crystalline.
3. A crystalline polymorph of Fosgonimeton mono sodium salt according to Clause 2, designated Form N2, which is characterized by data selected from one or more of the following:
   (i) an X-ray powder diffraction pattern substantially as depicted in FIG. 3;
   (ii) an X-ray powder diffraction pattern having peaks at 8.6, 9.3 and 16.4 degrees 2-theta±0.2 degrees 2-theta;
   (iii) a solid state ¹³C NM/JR spectrum having characteristic peaks at 178.4, 136.3, 54.5, 24.1 and 16.5 ppm±0.2 ppm;
   (iv) a solid state ¹³C NM/IR spectrum having the following chemical shift absolute differences from reference peak at 16.5 ppm±0.2 ppm: 167.4, 125.3, 43.5, 13.1 and 5.5 ppm±0.1 ppm;
   (v) a solid state ¹³C NMR spectrum substantially as depicted in FIG. 8, 8A or 8B; and
   (vi) combinations of these data.
4. Crystalline Fosgonimeton mono sodium salt according to Clause 3, which is characterized by an X-ray powder diffraction pattern having peaks at 8.6, 9.3 and 16.4 degrees 2-theta±0.2 degrees 2-theta, and also having any one or both additional peaks selected from 17.1 and 18.1 degrees 2-theta±0.2 degrees 2-theta.
5. Crystalline Fosgonimeton mono sodium salt according to Clause 3 or Clause 4, which is characterized by an X-ray powder diffraction pattern having peaks at 8.6, 9.3, 16.4, 17.1 and 18.1 degrees 2-theta±0.2 degrees 2-theta.
6. Crystalline Fosgonimeton mono sodium salt according to any of Clauses 3, 4 or 5, which is characterized by an X-ray powder diffraction pattern having peaks at 8.6, 9.3, 16.4, 17.1 and 18.1 degrees 2-theta±0.2 degrees 2-theta, and also having any one, two, three, four or five additional peaks selected from 5.7, 14.3, 23.0, 24.6 and 26.0±0.2 degrees 2-theta±0.2 degrees 2-theta.
7. Crystalline Fosgonimeton mono sodium salt according to any of Clauses 3, 4, 5, or 6, which is characterized by an X-ray powder diffraction pattern having peaks at 5.7, 8.6, 9.3, 14.3, 16.4, 17.1, 18.1, 23.0, 24.6 and 26.0 degrees 2-theta±0.2 degrees 2-theta.
8. Crystalline Fosgonimeton mono sodium salt according to any of Clauses 3, 4, 5, 6, or 7, which is characterized by an X-ray powder diffraction pattern having an absence of peaks at: 4.5 to 5.0 degrees 2-theta±0.2 degrees 2-theta.
9. Crystalline Fosgonimeton mono sodium salt according to any of Clauses 3, 4, 5, 6, 7, or 8, which is characterized by an X-ray powder diffraction pattern having an absence of peaks at: 6.5 to 7.2 degrees 2-theta±0.2 degrees 2-theta.
10. Crystalline Fosgonimeton mono sodium salt according to any of Clauses 3, 4, 5, 6, 7, 8, or 9, which is characterized by an X-ray powder diffraction pattern having an absence of peaks at: 13.6 to 13.8 degrees 2-theta±0.2 degrees 2-theta.
11. Crystalline Fosgonimeton mono sodium salt according to any of Clauses 3, 4, 5, 6, 7, 8, 9, or 10, which is characterized by an X-ray powder diffraction pattern having an absence of peaks at: 14.8 to 15.0 degrees 2-theta±0.2 degrees 2-theta.
12. Crystalline Fosgonimeton sodium salt according to any of Clauses 1, 2, 3, 4, 5, 6, 7, 8, 9, 10, or 11, which is a hydrate.
13. Crystalline Fosgonimeton sodium salt according to any of Clauses 1, 2, 3, 4, 5, 6, 7, 8, 9, 10, 11, or 12, which is a monohydrate.
14. Crystalline Fosgonimeton sodium salt according to any of Clauses 1, 2, 3, 4, 5, 6, 7, 8, 9, 10, 11, 12, or 13, which contains from about 2% to about 4% of water, preferably about 3% water, and particularly about 3.2% of water.
15. A process for preparing crystalline Fosgonimeton sodium salt Form N2 as defined in any of Clauses 3, 4, 5, 6, 7, 8, 9, 10, 11, 12, 13, or 14, which comprises:
   (i) suspending Fosgonimeton mono sodium salt in a solvent mixture comprising water and an organic solvent which is an alcohol (preferably a C₁-C₆ alcohol, more preferably a C₁-C₃ alcohol, and most preferably methanol or isopropanol) wherein the alcohol is used in an amount of: about 4 ml to about 12 ml, about 6 ml to about 11 ml, about 7 ml to about 10 ml, per gram of Fosgonimeton mono sodium salt; or suspending Fosgonimeton mono sodium salt in a solvent mixture comprising water and an organic solvent selected from: an ether (preferably a C₄-C₈ ether, more preferably a C₄-C₆ ether, particularly tetrahydrofuran or 2-methyltetrahydrofuran and most preferably 2-methyltetrahydrofuran), or an ester (preferably a C₄-C₈ ester, more preferably a C₄-C₆ ester, and particularly ethyl acetate), preferably wherein the ether or ester is used in an amount of: about 4 ml to about 20 ml, about 5 ml to about 15 ml, about 6 ml to about 11 ml, about 7 ml to about 10 ml, about 8 ml to about 10 ml, or about 9 ml to about 10 ml, or about 9.7 ml or about 10 ml per gram of Fosgonimeton mono sodium salt;
   (ii) optionally stirring the suspension; and
   (iii) optionally isolating the crystalline Fosgonimeton.
16. A process according to Clause 15, wherein the solvent mixture in step (i) comprises: water and methanol, water and isopropanol, water and 2-methyltetrahydrofuran, or water and ethyl acetate.
17. A process according to Clause 15 or Clause 16, wherein water is used in an amount of: about 8% to about 15%, about 8% to about 13%, about 8% to about 12%, about 9% to about 11%, or about 10%, by volume relative to the solvent mixture.
18. A process according to any of Clauses 15, 16 or 17, wherein step (i) is carried out at a temperature of: about 18° C. to about 30° C., about 20° C. to about 30° C., about 22° C. to about 27° C., about 20° C. to about 25° C., or about 25° C.
19. A process according to any of Clauses 15, 16, 17, or 18, wherein step (ii) is carried out, preferably at a temperature of: about 18° C. to about 30° C., about 20° C. to about 30° C., about 22° C. to about 27° C., about 20° C. to about 25° C., or about 25° C.
20. A process according to any of Clauses 15, 16 17, 18, or 19, wherein the suspension of Fosgonimeton sodium salt in step (i) is prepared by a process comprising:
   (a) providing a suspension of Fosgonimeton in the organic solvent to form a suspension; and
   (b) combining the suspension with an aqueous solution of a base selected from sodium carbonate, sodium hydrogen carbonate, or sodium hydroxide, preferably sodium carbonate or sodium hydroxide.
21. A process according to Clause 20, wherein step (b) comprises addition of the aqueous solution of a base to the suspension of Fosgonimeton in step (a), preferably wherein the addition is dropwise.
22. A process according to Clause 20 or Clause 21, wherein the base is sodium hydrogen carbonate or sodium hydroxide, and wherein the base is used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton.
23. A process according to any of Clause 20, 21 or 22, wherein the base is sodium carbonate, and wherein the base is used in an amount of: about 0.4 to about 4, about 0.5 to about 2, about 0.5 to about 1.5, about 0.5 to about 1.0, about 0.5 to about 0.8; or about 0.5 to about 0.6 mole equivalents relative to Fosgonimeton.
24. A process according to any of Clauses 20, 21, 22, or 23, wherein step (b) is carried out at a temperature of: about 18° C. to about 30° C., about 20° C. to about 30° C., about 22° C. to about 27° C., about 20° C. to about 25° C., or about 25° C.
25. A process according to any of Clauses 20, 21, 22, 23, or 24, wherein step (b) is carried out for a period of: about 1 hour to about 12 days, about 2 hours to about 10 days, about 3 hours to about 8 days, about 4 hours to about 7 days.
26. A process according to any of Clauses 20, 21, 22, 23, 24, or 25, wherein the Fosgonimeton in step (a) is Form 1 as defined in any of Clauses 90 to 101.
27. A process according to any of Clauses 15, 16, 17, 18, 19, 20, 21, 22, 23, 24, 25, or 26, wherein step (ii) is carried out, preferably by filtration, decantation or centrifuge, more preferably by filtration.
28. A process according to any of Clauses 15, 16, 17, 18, 19, 20, 21, 22, 23, 24, 25, 26, or 27, further comprising combining the Fosgonimeton mono sodium salt with at least one pharmaceutically acceptable excipient to form a pharmaceutical composition.
29. A product obtainable by a process according to any of Clauses 15, 16, 17, 18, 19, 20, 21, 22, 23, 24, 25, 26, 27, or 28.
30. A crystalline polymorph of Fosgonimeton mono sodium salt according to Clause 2, designated Form N5, which is characterized by data selected from:
   (i) an X-ray powder diffraction pattern substantially as depicted in FIG. 5; or
   (ii) an X-ray powder diffraction pattern having peaks at 13.8, 21.4 and 27.7 degrees 2-theta±0.2 degrees 2-theta.
31. Crystalline Fosgonimeton mono sodium salt according to Clause 30, which is characterized by an X-ray powder diffraction pattern having peaks at 13.8, 21.4 and 27.7 degrees 2-theta±0.2 degrees 2-theta, and also having any one or both additional peaks selected from 10.3 and 19.6 degrees 2-theta±0.2 degrees 2-theta.
32. Crystalline Fosgonimeton mono sodium salt according to Clause 30 or Clause 31, which is characterized by an X-ray powder diffraction pattern having peaks at 10.3, 13.8, 19.6, 21.4 and 27.7 degrees 2-theta±0.2 degrees 2-theta.
33. Crystalline Fosgonimeton mono sodium salt according to any of Clauses 31, 31 or 32, which is characterized by an X-ray powder diffraction pattern having peaks at 10.3, 13.8, 19.6, 21.4 and 27.7 degrees 2-theta±0.2 degrees 2-theta, and also having any one, two or three additional peaks selected from 11.6, 12.3 and 24.8±0.2 degrees 2-theta±0.2 degrees 2-theta.
34. Crystalline Fosgonimeton mono sodium salt according to any of Clauses 30, 31. 32, or 33, which is characterized by an X-ray powder diffraction pattern having peaks at 10.3, 11.6, 12.3, 13.8, 19.6, 21.4, 24.8 and 27.7 degrees 2-theta±0.2 degrees 2-theta.
35. Crystalline Fosgonimeton mono sodium salt according to any of Clauses 30, 31, 32, 33, or 34, which is characterized by an X-ray powder diffraction pattern having an absence of peaks at: 3.6 to 5.4 degrees 2-theta±0.2 degrees 2-theta.
36. Crystalline Fosgonimeton mono sodium salt according to any of Clauses 30, 31, 32, 33, 34, or 35, which is characterized by an X-ray powder diffraction pattern having an absence of peaks at: 9.2 to 9.8 degrees 2-theta±0.2 degrees 2-theta.
37. Crystalline Fosgonimeton mono sodium salt according to any of Clauses 30, 31, 32, 33, 34, 35, or 36, which is characterized by an X-ray powder diffraction pattern having an absence of peaks at: 10.7 to 11.0 degrees 2-theta±0.2 degrees 2-theta.
38. Crystalline Fosgonimeton mono sodium salt according to any of Clauses 30, 31, 32, 33, 34, 35, 36, or 37, which is characterized by an X-ray powder diffraction pattern having an absence of peaks at: 12.8 to 13.2 degrees 2-theta±0.2 degrees 2-theta.
39. Crystalline Fosgonimeton mono sodium salt according to any of Clauses 30, 31, 32, 33, 34, 35, 36, 37, or 38, which is characterized by an X-ray powder diffraction pattern having an absence of peaks at: 14.4 to 16.0 degrees 2-theta±0.2 degrees 2-theta.
40. Crystalline Fosgonimeton mono sodium salt according to any of Clauses 30, 31, 32, 33, 34, 35, 36, 37, 38, or 39, which is characterized by an X-ray powder diffraction pattern having an absence of peaks at: 30.2 to 31.6 degrees 2-theta±0.2 degrees 2-theta.
41. Crystalline Fosgonimeton mono sodium salt according to any of Clauses 30, 31, 32, 33, 34, 35, 36, 37, 38, 39, or 40, which is a hydrate.
42. Crystalline Fosgonimeton sodium salt according to any of Clauses 30, 31, 32, 33, 34, 35, 36, 37, 38 39, 40, or 41, which is a monohydrate.
43. Crystalline Fosgonimeton sodium salt according to any of Clauses 30, 31, 32, 33, 34, 35, 36, 37, 38 39, 40, 41, or 42, which contains from about 2% to about 4% of water, preferably about 3% water, and particularly about 3.2% of water.
44. Fosgonimeton sodium salt according to any of Clauses 1, 2, 3, 4, 5, 6, 7, 8, 9, 10, 11, 12, 13, 14, 29, 30, 31, 32, 33, 34, 35, 36, 37, 38, 39, 40, 41, 42, or 43, which is isolated.
45. Fosgonimeton sodium salt according to any of Clauses 3, 4, 5, 6, 7, 8, 9, 10, 11, 12, 13, 14, 29, 30, 31, 32, 33, 34, 35, 36, 37, 38, 39, 40, 41, 42, 43, or 44, which is polymorphically pure.
46. A process for preparing Fosgonimeton mono sodium salt Form N5 as defined in any of Clauses 30, 31, 32, 33, 34, 35, 36, 37, 38, 39, 40, 41, 42, 43, 44, or 45, which comprises:
   (i) suspending Fosgonimeton mono sodium salt in a solvent mixture comprising water and an organic solvent which is an alcohol (preferably a C₁-C₆ alcohol, more preferably a C₁-C₃ alcohol, and most preferably methanol) wherein the alcohol is used in an amount of: about 15 ml to about 30 ml, about 15 ml to about 28 ml, about 16 ml to about 25 ml, about 16 ml to about 22 ml, about 17 ml to about 21 ml, about 18 ml to about 20 ml, or about 19 or 19.2 ml, per gram of Fosgonimeton mono sodium salt;
   (ii) optionally stirring the suspension; and
   (iii) optionally isolating the crystalline Fosgonimeton.
47. A process according to Clause 46, wherein the solvent mixture in step (i) comprises: water and methanol.
48. A process according to Clause 46 or Clause 47, wherein water is used in an amount of: about 2% to about 7%, about 2% to about 6%, about 3% to about 6%, about 3% to about 5%, or about 4%, by volume relative to the solvent mixture.
49. A process according to any of Clauses 46, 47 or 48, wherein step (i) is carried out at a temperature of: about 18° C. to about 30° C., about 20° C. to about 30° C., about 22° C. to about 27° C., about 20° C. to about 25° C., or about 25° C.
50. A process according to any of Clauses 46, 47, 48, or 49, wherein step (ii) is carried out, preferably at a temperature of: about 18° C. to about 30° C., about 20° C. to about 30° C., about 22° C. to about 27° C., about 20° C. to about 25° C., or about 25° C.
51. A process according to any of Clauses 46, 47, 48, 49, or 50, wherein the suspension of Fosgonimeton sodium salt in step (i) is prepared by a process comprising:
   (a) providing a suspension of Fosgonimeton in the organic solvent to form a suspension; and
   (b) combining the suspension with an aqueous solution of a base selected from sodium carbonate, sodium hydrogen carbonate, or sodium hydroxide, preferably sodium carbonate or sodium hydroxide.
52. A process according to Clause 51, wherein step (b) comprises addition of the aqueous solution of a base to the suspension of Fosgonimeton in step (a), preferably wherein the addition is dropwise.
53. A process according to Clause 51 or Clause 52, wherein the base is sodium hydrogen carbonate or sodium hydroxide, and wherein the base is used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton.
54. A process according to any of Clauses 51, 52 or 53, wherein the base is sodium carbonate, and wherein the base is used in an amount of: about 1 to about 4, about 1 to about 3, or about 1 to about 2, mole equivalents relative to Fosgonimeton.
55. A process according to any of Clauses 51, 52, 53, or 54, wherein step (b) is carried out at a temperature of: about 18° C. to about 30° C., about 20° C. to about 30° C., about 22° C. to about 27° C., about 20° C. to about 25° C., or about 25° C.
56. A process according to any of Clauses 51, 52, 53, 54, or 55, wherein step (b) is carried out for a period of: about 4 hours to about 7 days, about 8 hours to about 5 days, about 12 hours to about 4 days, about 18 hours to about 3 days, or about 1 to about 2 days.
57. A process according to any of Clauses 51, 52, 53, 54, 55, or 56, wherein the Fosgonimeton in step (a) is Form 1 as defined in any of Clauses 90 to 101.
58. A process according to any of Clauses 46, 47, 48, 49, 50, 51, 52, 53, 54, 55, 56, or 57, wherein step (iii) is carried out, preferably by filtration, decantation or centrifuge, more preferably by filtration.
59. A process according to any of Clauses 46, 47, 48, 49, 50, 51, 52, 53, 54, 55, 56, 57, or 58, further comprising combining the Fosgonimeton mono sodium salt with at least one pharmaceutically acceptable excipient to form a pharmaceutical composition.
60. A product obtainable by a process according to any of Clauses 46, 47, 48, 49, 50, 51, 52, 53, 54, 55, 56, 57, 58, or 59.
61. Fosgonimeton potassium salt, preferably Fosgonimeton mono potassium salt.
62. Fosgonimeton potassium salt, preferably Fosgonimeton mono potassium salt according to Clause 61, which is crystalline.
63. A crystalline polymorph of Fosgonimeton mono potassium salt according to Clause 62, designated Form K1, which is characterized by data selected from:
   (i) an X-ray powder diffraction pattern substantially as depicted in FIG. 2A or 2B; or
   (ii) an X-ray powder diffraction pattern having peaks at 5.9, 8.8 and 23.6 degrees 2-theta±0.2 degrees 2-theta.
64. Crystalline Fosgonimeton mono potassium salt according to Clause 63, which is characterized by an X-ray powder diffraction pattern having peaks at 5.9, 8.8 and 23.6 degrees 2-theta±0.2 degrees 2-theta, and also having any one or both additional peaks selected from 18.3 and 20.6 degrees 2-theta±0.2 degrees 2-theta.
65. Crystalline Fosgonimeton mono potassium salt according to Clause 63 or Clause 64, which is characterized by an X-ray powder diffraction pattern having peaks at 5.9, 8.8, 18.3, 20.6 and 23.6±0.2 degrees 2-theta.
66. Crystalline Fosgonimeton mono potassium salt according to any of Clauses 63, 64 or 65, which is characterized by an X-ray powder diffraction pattern having peaks at 5.9, 8.8, 18.3, 20.6 and 23.6±0.2 degrees 2-theta±0.2 degrees 2-theta, and also having any one, two, three, four or five additional peaks selected from 10.9, 14.7, 15.6, 16.8 and 26.6±0.2 degrees 2-theta±0.2 degrees 2-theta.
67. Crystalline Fosgonimeton mono potassium salt according to any of Clauses 63, 64, 65, or 66, which is characterized by an X-ray powder diffraction pattern having peaks at 5.9, 8.8, 10.9, 14.7, 15.6, 16.8, 18.3, 20.6, 23.6, and 26.6 degrees 2-theta±0.2 degrees 2-theta.
68. Crystalline Fosgonimeton mono potassium salt according to any of Clauses 63, 64, 65, 66, or 67, which is characterized by an X-ray powder diffraction pattern having an absence of peaks at: 4.2 to 5.2 degrees 2-theta±0.2 degrees 2-theta.
69. Crystalline Fosgonimeton mono potassium salt according to any of Clauses 63, 64, 65, 66, 67, or 68, which is characterized by an X-ray powder diffraction pattern having an absence of peaks at: 6.5 to 8.0 degrees 2-theta±0.2 degrees 2-theta.
70. Crystalline Fosgonimeton mono potassium salt according to any of Clauses 63, 64, 65, 66, 67, 68, or 69, which is characterized by an X-ray powder diffraction pattern having an absence of peaks at: 9.4 to 10.2 degrees 2-theta±0.2 degrees 2-theta.
71. Fosgonimeton mono potassium salt according to any of Clauses 61, 62, 63, 64, 65, 66, 67, 68, 69, or 70, which is anhydrous.
72. Fosgonimeton potassium salt according to any of Clauses 61, 62, 63, 64, 65, 66, 67, 68, 69, 70, or 71, which is isolated.
73. Fosgonimeton potassium salt according to any of Clauses 61, 62, 63, 64, 65, 66, 67, 68, 69, 70, 71, or 72, which is polymorphically pure.
74. A process for preparing Fosgonimeton potassium salt Form K1 as defined in any of Clauses 63, 64, 65, 66, 67, 68, 69, 70, 71, 72, or 73, which comprises:
   (i) suspending Fosgonimeton mono potassium salt in a solvent mixture comprising water and an organic solvent selected from: an alcohol (preferably a C₁-C₆ alcohol, more preferably a C₁-C₃ alcohol, and most preferably methanol or isopropanol) or acetonitrile;
   (ii) optionally stirring the suspension; and
   (iii) optionally isolating the crystalline Fosgonimeton.
75. A process according to Clause 74, wherein the organic solvent is used in an amount of: about 4 ml to about 18 ml, about 5 ml to about 17 ml, about 6 ml to about 16 ml, about 7 ml to about 15 ml, about 8 ml to about 14 ml, or about 9 ml to about 13.5 ml, or about 9.4 or about 13.1 ml per gram of Fosgonimeton mono potassium salt.
76. A process according to Clause 74 or Clause 75, wherein the solvent mixture in step (i) comprises: water and methanol, water and isopropanol, or water and acetonitrile.
77. A process according to any of Clauses 74, 75 or 76, wherein water is used in an amount of: about 8% to about 20%, about 9% to about 18%, about 10% to about 17%, about 11% to about 16%, or about 11.8%, about 14.6%, or about 15.8%, by volume relative to the solvent mixture.
78. A process according to any of Clauses 74, 75, 76, or 77, wherein step (i) is carried out at a temperature of: about 18° C. to about 30° C., about 20° C. to about 30° C., about 22° C. to about 27° C., about 20° C. to about 25° C., or about 25° C.
79. A process according to any of Clauses 74, 75, 76, 77, or 78, wherein step (ii) is carried out, preferably at a temperature of: about 18° C. to about 30° C., about 20° C. to about 30° C., about 22° C. to about 27° C., about 20° C. to about 25° C., or about 25° C.
80. A process according to any of Clauses 74, 75, 76, 77, 78, or 79, wherein the suspension of Fosgonimeton potassium salt in step (i) is prepared by a process comprising:
   (a) providing a suspension of Fosgonimeton in the organic solvent to form a suspension; and
   (b) combining the suspension with an aqueous solution of a base selected from potassium carbonate, potassium hydrogen carbonate, or potassium hydroxide, preferably potassium hydroxide.
81. A process according to Clause 80, wherein step (b) comprises addition of the aqueous solution of a base to the suspension of Fosgonimeton in step (a), preferably wherein the addition is dropwise.
82. A process according to Clause 80 or Clause 81, wherein the base is potassium hydroxide, and wherein the base is used in an amount of: about 0.9 to about 4, about 1.0 to about 3, about 1.0 to about 2.0, about 1.0 to about 1.5, about 1.0 to about 1.2; or about 1.0 to about 1.1 mole equivalents relative to Fosgonimeton.
83. A process according to any of Clauses 80, 81 or 82, wherein step (b) is carried out at a temperature of: about 18° C. to about 30° C., about 20° C. to about 30° C., about 22° C. to about 27° C., about 20° C. to about 25° C., or about 25° C.
84. A process according to any of Clauses 80, 81, 82, or 83, wherein step (b) is carried out for a period of: about 1 hour to about 12 days, about 2 hours to about 10 days, about 3 hours to about 8 days, about 3 hours to about 7 days.
85. A process according to any of Clauses 80, 81, 82, 83, or 84, wherein the Fosgonimeton in step (a) is Form 1 as defined in any of Clauses 90 to 101.
86. A process according to any of Clauses 74, 75, 76, 77, 78, 79, 80, 81, 82, 83, 84, or 85, wherein step (iii) is carried out, preferably by filtration, decantation or centrifuge, more preferably by filtration.
87. A process according to any of Clauses 74, 75, 76, 77, 78, 79, 80, 81, 82, 83, 84, 85, or 86, further comprising combining the Fosgonimeton mono potassium salt with at least one pharmaceutically acceptable excipient to form a pharmaceutical composition.
88. A product obtainable by a process according to any of Clauses 74, 75, 76, 77, 78, 79, 80, 81, 82, 83, 84, 85, 86, or 87.
89. Crystalline Fosgonimeton.
90. Crystalline Fosgonimeton according to Clause 89, designated Form 1, which is characterized by data selected from one or more of the following:
   (i) an X-ray powder diffraction pattern substantially as depicted in FIG. 1;
   (ii) an X-ray powder diffraction pattern having peaks at 5.2, 9.4 and 12.9 degrees 2-theta±0.2 degrees 2-theta;
   (iii) a solid state ¹³C NMR spectrum having characteristic peaks at 171.5, 149.2, 117.7, 57.5 and 33.5 ppm±0.2 ppm;
   (iv) a solid state ¹³C NM/IR spectrum having the following chemical shift absolute differences from reference peak at 24.5 ppm±0.2 ppm: 147.0, 124.7, 93.2, 33.0 and 9.0 ppm±0.1 ppm;
   (v) a solid state ¹³C NMR spectrum substantially as depicted in FIG. 7, 7A or 7B; and
   (vi) combinations of these data.
91. Crystalline Fosgonimeton according to Clause 90, which is characterized by an X-ray powder diffraction pattern having peaks at 5.2, 9.4 and 12.9 degrees 2-theta±0.2 degrees 2-theta, and also having any one or both additional peaks selected from 13.7 and 18.9 degrees 2-theta±0.2 degrees 2-theta.
92. Crystalline Fosgonimeton according to Clause 90 or Clause 91, which is characterized by an X-ray powder diffraction pattern having peaks at 5.2, 9.4, 12.9, 13.7 and 18.9 degrees 2-theta±0.2 degrees 2-theta.
93. Crystalline Fosgonimeton according to any of Clauses 90, 91 or 92, which is characterized by an X-ray powder diffraction pattern having peaks at 5.2, 9.4, 12.9, 13.7 and 18.9 degrees 2-theta±0.2 degrees 2-theta, and also having any one, two, three, four or five additional peaks selected from 7.7, 11.1, 17.9, 19.8 and 22.3 degrees 2-theta±0.2 degrees 2-theta.
94. Crystalline Fosgonimeton according to any of Clauses 90, 91, 92, or 93, which is characterized by an X-ray powder diffraction pattern having peaks at 5.2, 7.7, 9.4, 11.1, 12.9, 13.7, 17.9, 18.9, 19.8 and 22.3 degrees 2-theta±0.2 degrees 2-theta.
95. Crystalline Fosgonimeton according to any of Clauses 90, 91, 92, 93, or 94, which is characterized by an X-ray powder diffraction pattern having an absence of peaks at either: 3.0 to 4.2 degrees 2-theta±0.2 degrees 2-theta, or 3.5 to 4.0 degrees 2-theta±0.2 degrees 2-theta.
96. Crystalline Fosgonimeton according to any of Clauses 90, 91, 92, 93, 94, or 95, which is characterized by an X-ray powder diffraction pattern having an absence of peaks at: 6.0 to 7.2 degrees 2-theta±0.2 degrees 2-theta.
97. Crystalline Fosgonimeton according to any of Clauses 90, 91, 92, 93, 94, 95, or 96, which is characterized by an X-ray powder diffraction pattern having an absence of peaks at: 8.2 to 8.6 degrees 2-theta±0.2 degrees 2-theta.
98. Crystalline Fosgonimeton according to any of Clauses 90, 91, 92, 93, 94, 95, 96, or 97, which is characterized by an X-ray powder diffraction pattern having an absence of peaks at: 16.4 to 17.0 degrees 2-theta±0.2 degrees 2-theta.
99. Crystalline Fosgonimeton according to any of Clauses 90, 91, 92, 93, 94, 95, 96, 97, or 98, which is anhydrous.
100. Crystalline Fosgonimeton according to any of Clauses 90, 91, 92, 93, 94, 95, 96, 97, 98, or 99, which is isolated.
101. Crystalline Fosgonimeton according to any of Clauses 90, 91, 92, 93, 94, 95, 96, 97, 98, 99, or 100, which is polymorphically pure.
102. A process for preparing crystalline Fosgonimeton Form 1 as defined in any of Clauses 90, 91, 92, 93, 94, 95, 96, 97, 98, 99, 100, or 101, which comprises crystallisation of Fosgonimeton in a solvent mixture comprising water and at least one organic solvent selected from: an alcohol (preferably a C₁-C₆ alcohol, more preferably a C₁-C₃ alcohol, and most preferably ethanol or methanol), an ether (preferably a C₄-C₈ ether, more preferably a C₄-C₆ ether, and most preferably tetrahydrofuran), or acetonitrile.
103. A process according to Clause 102, comprising crystallisation of Fosgonimeton from a solvent mixture selected from: ethanol and water, acetonitrile and water, methanol and water, or tetrahydrofuran and water.
104. A process according to Clause 102 or Clause 103, wherein the ratio of organic solvent to water is: about 3:1 to about 1:3, about 2.5:1 to about 1:2.5, about 2:1 to about 1:2, about 1.5:1 to about 1:1.5, about 1.2:1 to about 1:1.2, about 1.1:1 to about 1:1.1, or about 1:1.
105. A process according to any of Clauses 102, 103, or 104, wherein the organic solvent is used in an amount of: about 10 ml to about 100 ml, about 10 ml to about 80 ml, about 10 ml to about 70 ml, about 15 ml to about 65 ml, about 20 ml to about 60 ml, or about 20 ml to about 55 ml, or about 25 to about 50 ml per gram of Fosgonimeton.
106. A process according to any of Clauses 102, 103, 104, or 105, comprising:
   (i) providing a solution of Fosgonimeton in the solvent mixture;
   (ii) optionally cooling, and optionally stirring;
   (iii) optionally isolating; and
   (iv) optionally drying the crystalline Fosgonimeton.
107. A process according to Clause 106, wherein the solvent mixture in step (i) comprises: water and methanol, water and isopropanol, or water and acetonitrile, preferably wherein the ratio of water to the organic solvent is about 1:1.
108. A process according to Clause 106 or Clause 107, wherein step (i) is carried out at a temperature of: about 45° C. to about 100° C., about 50° C. to about 90° C., about 55° C. to about 80° C., about 60° C. to about 80° C., or about 68° C. to about 77° C.
109. A process according to any of Clauses 106, 107 or 108, wherein the cooling in step (ii) is carried out, preferably wherein the mixture is cooled to a temperature of: about −20° C. to about 30° C., about −18° C. to about 28° C., about −16° C. to about 25° C., or about −15° C. to about 25° C.
110. A process according to any of Clauses 106, 107, 108, or 109, wherein the stirring in step (ii) is carried out for period of: about 5 minutes to about 10 hours, about 10 minutes to about 6 hours, about 10 minutes to about 4 hours, about 10 minutes to about 2 hours, or about 10 minutes to about 1 hour.
111. A process according to any of Clauses 106, 107, 108, 109, or 110, wherein step (iii) is carried out, preferably by filtration, decantation or centrifuge, more preferably by filtration.
112. A process according to any of Clauses 106, 107, 108, 109, 110, or 111, further comprising combining the Fosgonimeton with at least one pharmaceutically acceptable excipient to form a pharmaceutical composition.
113. A product obtainable by a process according to any of Clauses 106, 107, 108, 109, 110, 111, or 112.
114. A pharmaceutical composition comprising a crystalline product according to any of Clauses 1-14, 29, 30-45, 60, 61-73, 88, 89-101, and 113, and at least one pharmaceutically acceptable excipient.
115. Use of a crystalline product according to any of Clauses 1-14, 29, 30-45, 60, 61-73, 88, 89-101, and 113, for the preparation of a pharmaceutical composition and/or formulation
116. A process for preparing the pharmaceutical composition according to Clause 113, comprising combining a crystalline product according to any of Clauses 1-14, 29, 30-45, 60, 61-73, 88, 89-101, and 113, with at least one pharmaceutically acceptable excipient.
117. A crystalline product according to any of Clauses 1-14, 29, 30-45, 60, 61-73, 88, 89-101, and 113, or a pharmaceutical composition according to Clause 114, for use as a medicament.
118. A crystalline product according to any of Clauses 1-14, 29, 30-45, 60, 61-73, 88, 89-101, and 113, or a pharmaceutical composition according to Clause 114, for use in the treatment of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and schizophrenia, and particularly Diabetic Peripheral Neuropathic pain or Post-Herpetic Neuralgia, or Alzheimer's Disease, Parkinson's disease (PD), Lewy body disease or disorder(s) (LBD), Parkinson's Disease Dementia (PDD) and/or dementia with Lewy bodies (DLB).
119. A method of treating Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression and schizophrenia, and particularly Diabetic Peripheral Neuropathic pain or Post-Herpetic Neuralgia, comprising administering a therapeutically effective amount of a crystalline product according to any of Clauses 1-14, 29, 30-45, 60, 61-73, 88, 89-101, and 113, or a pharmaceutical composition according to Clause 114, to a subject in need of the treatment.
120. Use of a crystalline product according to any of Clauses 1-14, 29, 30-45, 60, 61-73, 88, 89-101, and 113, in the preparation of another solid state form of Fosgonimeton, or another solid state form of a salt of Fosgonimeton, preferably Fosgonimeton sodium or Fosgonimeton potassium.
121. A process for preparing a solid state form of Fosgonimeton, of a salt of Fosgonimeton, preferably Fosgonimeton sodium or Fosgonimeton potassium, comprising preparing any one or a combination of a crystalline product according to any one of Clauses 1-14, 29, 30-45, 60, 61-73, 88, 89-101, and 113, and converting it to another a solid state form of Fosgonimeton or a salt thereof.

1. A crystalline polymorph of Fosgonimeton mono sodium salt designated Form N2, which is characterized by data selected from one or more of the following:
(i) an X-ray powder diffraction pattern substantially as depicted in FIG. 3;
(ii) an X-ray powder diffraction pattern having peaks at 8.6, 9.3 and 16.4 degrees 2-theta±0.2 degrees 2-theta;
(iii) a solid state ¹³C NMR spectrum having characteristic peaks at 178.4, 136.3, 54.5, 24.1 and 16.5 ppm±0.2 ppm;
(iv) a solid state ¹³C NMR spectrum having the following chemical shift absolute differences from reference peak at 16.5 ppm±0.2 ppm: 167.4, 125.3, 43.5, 13.1 and 5.5 ppm±0.1 ppm;
(v) a solid state ¹³C NMR spectrum substantially as depicted in FIG. 8, 8A or 8B; and
(vi) combinations of these data.

2. The crystalline polymorph of Fosgonimeton mono sodium salt according to claim 1, which is characterized by an X-ray powder diffraction pattern having peaks at 8.6, 9.3 and 16.4 degrees 2-theta±0.2 degrees 2-theta, and also having any one or both additional peaks selected from 17.1 and 18.1 degrees 2-theta±0.2 degrees 2-theta.

3. The crystalline polymorph of Fosgonimeton mono sodium salt according to claim 1, which is characterized by an X-ray powder diffraction pattern having peaks at 8.6, 9.3, 16.4, 17.1 and 18.1 degrees 2-theta±0.2 degrees 2-theta.

4. The crystalline polymorph of Fosgonimeton mono sodium salt according to claim 1, which is characterized by an X-ray powder diffraction pattern having peaks at 8.6, 9.3, 16.4, 17.1 and 18.1 degrees 2-theta±0.2 degrees 2-theta, and also having any one, two, three, four or five additional peaks selected from 5.7, 14.3, 23.0, 24.6 and 26.0±0.2 degrees 2-theta±0.2 degrees 2-theta.

5. The crystalline polymorph of Fosgonimeton mono sodium salt according to claim 1, which is characterized by an X-ray powder diffraction pattern having peaks at 5.7, 8.6, 9.3, 14.3, 16.4, 17.1, 18.1, 23.0, 24.6 and 26.0 degrees 2-theta±0.2 degrees 2-theta.

6. A crystalline polymorph of Fosgonimeton mono sodium salt, designated Form N5, which is characterized by data selected from:
(i) an X-ray powder diffraction pattern substantially as depicted in FIG. 5; or
(ii) an X-ray powder diffraction pattern having peaks at 13.8, 21.4 and 27.7 degrees 2-theta±0.2 degrees 2-theta.

7. The crystalline polymorph of Fosgonimeton mono sodium salt according to claim 6, which is characterized by an X-ray powder diffraction pattern having peaks at 13.8, 21.4 and 27.7 degrees 2-theta±0.2 degrees 2-theta, and also having any one or both additional peaks selected from 10.3 and 19.6 degrees 2-theta±0.2 degrees 2-theta.

8. The crystalline polymorph of Fosgonimeton mono sodium salt according to claim 6, which is characterized by an X-ray powder diffraction pattern having peaks at 10.3, 13.8, 19.6, 21.4 and 27.7 degrees 2-theta±0.2 degrees 2-theta.

9. The crystalline polymorph of Fosgonimeton mono sodium salt according to claim 6, which is characterized by an X-ray powder diffraction pattern having peaks at 10.3, 13.8, 19.6, 21.4 and 27.7 degrees 2-theta±0.2 degrees 2-theta, and also having any one, two or three additional peaks selected from 11.6, 12.3 and 24.8±0.2 degrees 2-theta±0.2 degrees 2-theta.

10. The crystalline polymorph of Fosgonimeton mono sodium salt according to claim 6, which is characterized by an X-ray powder diffraction pattern having peaks at 10.3, 11.6, 12.3, 13.8, 19.6, 21.4, 24.8 and 27.7 degrees 2-theta±0.2 degrees 2-theta.

11. The crystalline polymorph of Fosgonimeton mono sodium salt according to claim 1, which is isolated.

12. The crystalline polymorph of Fosgonimeton mono sodium salt according to claim 1, which is polymorphically pure.

13. A Fosgonimeton potassium salt, which is a Fosgonimeton mono potassium salt.

14. The Fosgonimeton potassium salt according to claim 13, wherein the Fosgonimeton mono potassium salt is crystalline.

15. A crystalline polymorph of the Fosgonimeton mono potassium salt according to claim 14, designated Form K1, which is characterized by data selected from:
(i) an X-ray powder diffraction pattern substantially as depicted in FIG. 2A or 2B; or
(ii) an X-ray powder diffraction pattern having peaks at 5.9, 8.8 and 23.6 degrees 2-theta±0.2 degrees 2-theta.

16. The crystalline polymorph of the Fosgonimeton mono potassium salt according to claim 15, which is characterized by an X-ray powder diffraction pattern having peaks at 5.9, 8.8 and 23.6 degrees 2-theta±0.2 degrees 2-theta, and also having any one or both additional peaks selected from 18.3 and 20.6 degrees 2-theta±0.2 degrees 2-theta.

17. The crystalline polymorph of the Fosgonimeton mono potassium salt according to claim 15, which is characterized by an X-ray powder diffraction pattern having peaks at 5.9, 8.8, 18.3, 20.6 and 23.6±0.2 degrees 2-theta.

18. The crystalline polymorph of the Fosgonimeton mono potassium salt according to claim 15, which is characterized by an X-ray powder diffraction pattern having peaks at 5.9, 8.8, 18.3, 20.6 and 23.6±0.2 degrees 2-theta±0.2 degrees 2-theta, and also having any one, two, three, four or five additional peaks selected from 10.9, 14.7, 15.6, 16.8 and 26.6±0.2 degrees 2-theta±0.2 degrees 2-theta.

19. The crystalline polymorph of the Fosgonimeton mono potassium salt according to claim 15, which is characterized by an X-ray powder diffraction pattern having peaks at 5.9, 8.8, 10.9, 14.7, 15.6, 16.8, 18.3, 20.6, 23.6, and 26.6 degrees 2-theta±0.2 degrees 2-theta.

20. The Fosgonimeton potassium salt according to claim 13, which is isolated.

21. The Fosgonimeton potassium salt according to claim 15, which is polymorphically pure.

22. A crystalline Fosgonimeton, designated Form 1, which is characterized by data selected from one or more of the following:
(i) an X-ray powder diffraction pattern substantially as depicted in FIG. 1;
(ii) an X-ray powder diffraction pattern having peaks at 5.2, 9.4 and 12.9 degrees 2-theta±0.2 degrees 2-theta;
(iii) a solid state ¹³C NMR spectrum having characteristic peaks at 171.5, 149.2, 117.7, 57.5 and 33.5 ppm±0.2 ppm;
(iv) a solid state ¹³C NMR spectrum having the following chemical shift absolute differences from reference peak at 24.5 ppm±0.2 ppm: 147.0, 124.7, 93.2, 33.0 and 9.0 ppm±0.1 ppm;
(v) a solid state ¹³C NMR spectrum substantially as depicted in FIG. 7, 7A or 7B; and
(vi) combinations of these data.

23. The crystalline Fosgonimeton according to claim 22, which is characterized by an X-ray powder diffraction pattern having peaks at 5.2, 9.4 and 12.9 degrees 2-theta±0.2 degrees 2-theta, and also having any one or both additional peaks selected from 13.7 and 18.9 degrees 2-theta±0.2 degrees 2-theta.

24. The crystalline Fosgonimeton according to claim 22, which is characterized by an X-ray powder diffraction pattern having peaks at 5.2, 9.4, 12.9, 13.7 and 18.9 degrees 2-theta±0.2 degrees 2-theta.

25. The crystalline Fosgonimeton according to claim 22, which is characterized by an X-ray powder diffraction pattern having peaks at 5.2, 9.4, 12.9, 13.7 and 18.9 degrees 2-theta±0.2 degrees 2-theta, and also having any one, two, three, four or five additional peaks selected from 7.7, 11.1, 17.9, 19.8 and 22.3 degrees 2-theta±0.2 degrees 2-theta.

26. The crystalline Fosgonimeton according to claim 22, which is characterized by an X-ray powder diffraction pattern having peaks at 5.2, 7.7, 9.4, 11.1, 12.9, 13.7, 17.9, 18.9, 19.8 and 22.3 degrees 2-theta±0.2 degrees 2-theta.

27. The crystalline Fosgonimeton according to claim 22, which is isolated.

28. The crystalline Fosgonimeton according to claim 22, which is polymorphically pure.

29. A pharmaceutical composition comprising the crystalline Fosgonimeton according to claim 1, and at least one pharmaceutically acceptable excipient.

30. (canceled)

31. A process for preparing a pharmaceutical composition, comprising combining the crystalline Fosgonimeton according to claim 1, with at least one pharmaceutically acceptable excipient.

32. (canceled)

33. (canceled)

34. A method of treating at least one of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression, schizophrenia, Diabetic Peripheral Neuropathic pain, or Post-Herpetic Neuralgia, comprising administering a therapeutically effective amount of the crystalline Fosgonimeton according to claim 1, to a subject in need of the treatment.

35. (canceled)

36. A process for preparing a solid state form of Fosgonimeton, of a salt of Fosgonimeton, including at least one of Fosgonimeton sodium or Fosgonimeton potassium, the process comprising preparing the crystalline Fosgonimeton according to claim 1, and converting the crystalline Fosgonimeton to another a solid state form of Fosgonimeton or a salt thereof.

37. A pharmaceutical composition comprising the crystalline polymorph of Fosgonimeton according to claim 6, and at least one pharmaceutically acceptable excipient.

38. A process for preparing a pharmaceutical composition, comprising combining the crystalline polymorph of Fosgonimeton according to claim 6, with at least one pharmaceutically acceptable excipient.

39. A method of treating at least one of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression, schizophrenia, Diabetic Peripheral Neuropathic pain, or Post-Herpetic Neuralgia, comprising administering a therapeutically effective amount of the crystalline polymorph of Fosgonimeton according to claim 6, to a subject in need of the treatment.

40. A process for preparing a solid state form of Fosgonimeton, of a salt of Fosgonimeton, including at least one of Fosgonimeton sodium or Fosgonimeton potassium, the process comprising preparing the crystalline polymorph of Fosgonimeton according to claim 6, and converting the crystalline Fosgonimeton to another a solid state form of Fosgonimeton or a salt thereof.

41. A pharmaceutical composition comprising the Fosgonimeton potassium salt according to claim 13, and at least one pharmaceutically acceptable excipient.

42. A process for preparing a pharmaceutical composition, comprising combining the Fosgonimeton potassium salt according to claim 13, with at least one pharmaceutically acceptable excipient.

43. A method of treating at least one of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression, schizophrenia, Diabetic Peripheral Neuropathic pain, or Post-Herpetic Neuralgia, comprising administering a therapeutically effective amount of the Fosgonimeton potassium salt according to claim 13, to a subject in need of the treatment.

44. A process for preparing a solid state form of Fosgonimeton, of a salt of Fosgonimeton, including at least one of Fosgonimeton sodium or Fosgonimeton potassium, the process comprising preparing the Fosgonimeton potassium salt according to claim 13, and converting a crystalline Fosgonimeton of the Fosgonimeton potassium salt to another a solid state form of Fosgonimeton or a salt thereof.

45. A pharmaceutical composition comprising the crystalline Fosgonimeton according to claim 22, and at least one pharmaceutically acceptable excipient.

46. A process for preparing a pharmaceutical composition, comprising combining the crystalline Fosgonimeton according to claim 22, with at least one pharmaceutically acceptable excipient.

47. A method of treating at least one of Diabetic Peripheral Neuropathic pain, Post-Herpetic Neuralgia, Dementia, cognitive impairment, Alzheimer's Disease, Lewy Body Disease or disorders, Parkinson's Disease, Parkinson's Disease Dementia, Dementia with Lewy bodies, depression, schizophrenia, Diabetic Peripheral Neuropathic pain, or Post-Herpetic Neuralgia, comprising administering a therapeutically effective amount of the crystalline Fosgonimeton according to claim 22, to a subject in need of the treatment.

48. A process for preparing a solid state form of Fosgonimeton, of a salt of Fosgonimeton, including at least one of Fosgonimeton sodium or Fosgonimeton potassium, the process comprising preparing the crystalline Fosgonimeton according to claim 22, and converting the crystalline Fosgonimeton to another a solid state form of Fosgonimeton or a salt thereof.
